(19) United States
(12) Patent Application Publication
(10) Pub. No.: US 2025/0275833 A1
(43) Pub. Date: Sep. 4, 2025

(54) PROCESSING 2D INTRAORAL IMAGES AND RENDERING NOVEL VIEWS OF PATIENTS

(71) Applicant: ALIGN TECHNOLOGY, INC., San Jose, CA (US)

(72) Inventors: Michael SEEBER, Zurich (CH); Christopher E. CRAMER, Durham, NC (US); Niko Benjamin HUBER, Zug (CH); Phillip Thomas HARRIS, Cary, NC (US); Guotu LI, Apex, NC (US); Michael CHANG, Zurich (CH); Chad Clayton BROWN, Cary, NC (US); Chao SHI, Morrisville, NC (US)

(73) Assignee: ALIGN TECHNOLOGY, INC., San Jose, CA (US)

(21) Appl. No.: 19/069,092

(22) Filed: Mar. 3, 2025

Related U.S. Application Data
(60) Provisional application No. 63/561,123, filed on Mar. 4, 2024.

(51) Int. Cl. A61C 7/00; G06T 7/00; G06T 11/00

(52) U.S. Cl. CPC A61C 7/002; G06T 7/0016; G06T 11/00; G06T 2207/10016; G06T 2207/30036; G06T 2210/41

(57) ABSTRACT

Systems, apparatuses, and methods disclosed herein are directed to systems, methods, and apparatuses for enabling a patient to capture intra-oral images and for the processing of those images to assist a dentist or orthodontist in evaluating the state of a patient's teeth and the development or progress of a treatment plan.

### RELATED APPLICATIONS

This application claims the benefit under 35 U.S.C. § 119 (e) of U.S. Provisional Patent Application No. 63/561,123, filed Mar. 4, 2024, which is incorporated, in its entirety, by this reference.

### BACKGROUND

Intra-oral images are used to assist a dentist or orthodontist in determining the conditions in a patient's mouth, including tooth placement, tooth alignment, and gum conditions. Intra-oral images can be helpful in evaluating overall dental health and monitoring the progress of a treatment plan designed to move specific teeth into a desired position and alignment.

However, conventional approaches to capturing and using such images suffer from several disadvantages. These include the need for a patient to visit a dentist's (or orthodontist's) office so that the images can be obtained. A possible solution to this problem is asking a patient to provide images that are captured using a mobile phone or similar device, thereby eliminating the need for a physical presence in a dentist's office.

While this is more convenient for the patient, the quality of the captured images may suffer and, in some cases, render the images unsuitable for use. One approach to overcoming this problem is the use of a cheek retractor, which may be used to pull a patient's cheek and lips away from their teeth so that images more clearly show their teeth. The patient is then asked to take multiple photos of their teeth from different perspectives and provide those to the dentist for evaluation.

Unfortunately, this approach also has disadvantages in that monitoring a treatment plan to adjust one or more of the patient's teeth requires consistent and relatively high-quality images. This may be difficult to achieve by a patient using their own device due to lack of a common viewpoint for image capture (i.e., one based on a consistent or known camera position relative to the mouth, camera rotation or other perspective changes during image capture, or alignment of the camera optics to the mouth) and varying image capture conditions (such as differences in ambient lighting, camera focus, camera or patient movement, or other camera settings). Furthermore, the current capture process typically involves single-image acquisition, thereby precluding the possibility of fusing information from multiple images to mitigate temporal issues that may detrimentally affect image quality.

The possible variations in camera position, camera settings, and environmental conditions during attempts to capture images may render images unfeasible for use and/or unable to be used to determine the information needed by the dentist. This is because a dentist may need to know actual tooth position (as opposed to an apparent position impacted by camera motion or differences in camera position during image captures), actual gum tissue color, or other indications of the current state of a patient's teeth and gums. Further, use of automated image processing techniques typically requires consistent image capture conditions, or at least a set of known parameters describing those conditions so that images can be adjusted or normalized prior to being evaluated.

What is desired are systems, apparatuses, and methods for more effectively enabling a patient to capture intra-oral images and for the processing of those images to assist a dentist or orthodontist in evaluating the state of a patient's teeth and the development or progress of a treatment plan. Embodiments of the disclosure address this and other objectives both individually and collectively.

### SUMMARY

In some embodiments, the systems, apparatuses, and methods disclosed and/or described herein are directed to systems, methods, and apparatuses for enabling a patient to capture intra-oral images (e.g., photos, video) and for the processing of those images and video to assist a dentist, orthodontist, or other dental professions in evaluating the state of a patient's dentition, including their teeth, their bite, chewing motion, and jaw articulation and the development or progress of a treatment plan. In one embodiment, the captured images are derived from a video segment obtained by a patient.

In some embodiments, the captured images are used as the basis for developing a reconstructed representation of the current state of a patient's teeth by “fusing” information obtained from multiple images. The reconstruction can be used to generate novel images of a specified area of the mouth and/or images taken from a desired perspective or environmental condition. This enables a dentist to better determine the current state of the patient's teeth and to evaluate and/or refine a treatment plan. The described and/or disclosed approach also provides a capability for a dentist or orthodontist to have a consistent view of the patient's teeth when monitoring a treatment plan over time.

In one embodiment, as part of developing the reconstructed or fused representation, the images provided by a patient may be evaluated to determine if they are suitable for use in developing the reconstructed representation. In one embodiment, this evaluation may involve image quality assessment, filtering based on one or more characteristics, combining one or more images, or determining if an image or images are able to be classified by a trained model.

In one embodiment, the disclosure is directed to a method for capturing intra-oral images of a patient, processing the captured images to generate a reliable and consistent image or set of images of the patient's teeth and gums, and using the generated image or images to evaluate the current state of the patient's teeth and/or gums. From that information, the progress in moving the teeth to a desired position and alignment may be determined.

The disclosed and/or described image capture and processing techniques enable a patient to use a mobile device to capture a set of images, either directly or from a video, and provide those to a dentist or orthodontist for use in developing or monitoring the progress of a treatment plan intended to move the patient's teeth to a desired position and alignment.

In some embodiments, the dentist or orthodontist may receive the captured images or video, generate a set of images from the video, apply one or more image processing techniques to evaluate the suitability of using the images, and then use one or more suitable images as inputs to a process to reconstruct a representation of the patient's mouth and teeth. The reconstructed representation may then be used to generate additional images depicting the patient's teeth from a desired or baseline perspective to assist the dentist or orthodontist in determining the current state of the teeth, and from that, the next step in or changes to a treatment plan.

Images provided by the patient and/or generated from the reconstructed representation may also be used as inputs for purposes of training the model or as inputs to a trained model that operates as a classifier to identify an aspect of a patient's mouth or teeth. Such a model may be used for a diagnostic, monitoring, or visualization function based on intra-oral images, where such models benefit from a consistent, high-quality data source.

In some embodiments, the images may be captured video or videos, which may be used as the basis for developing a reconstructed representation of the current state of a patient's teeth by “fusing” information obtained from the video or videos. The reconstruction can be used to generate novel, videos of a specified area of the mouth and/or videos taken from a desired perspective or environmental condition. This enables a dentist to better determine the current state of the patient's dentition and to evaluate and/or refine a treatment plan. The described and/or disclosed approach also provides a capability for a dentist or orthodontist to have a consistent view of the patient's dentition when monitoring a treatment plan over time.

In one embodiment, as part of developing the reconstructed or fused representation, the videos provided by a patient may be evaluated to determine if they are suitable for use in developing the reconstructed representation. In one embodiment, this evaluation may involve image quality assessment of the video, filtering the video based on one or more characteristics, combining one or more videos, or determining if a video or videos are able to be classified by a trained model.

In one embodiment, the disclosure is directed to a method for capturing intra-oral video of a patient, processing the captured video to generate a reliable and consistent video or set of videos of the patient's intraoral cavity, and using the generated video or videos to evaluate the current state of the patient's intraoral cavity, including, teeth, bite, chewing motion, jaw articulation, and other aspects of oral health. From that information, the progress in moving the teeth to a desired position and alignment, jaw articulation or position, etc. may be determined.

The disclosed and/or described video capture and processing techniques enable a patient to use a mobile device to capture a video or videos, and provide those to a dentist or orthodontist for use in developing or monitoring the progress of a treatment plan.

In some embodiments, the dentist or orthodontist may receive the captured video, apply one or more image processing techniques to evaluate the suitability of using the video, such as each frame of the video, and then use one or more suitable videos, portions of videos, etc. as inputs to a process to reconstruct a moving representation of the patient's intraoral cavity. The reconstructed representation may then be used to generate additional videos depicting the patient's intraoral cavity from a desired or baseline perspective to assist the dentist or orthodontist in determining the current state of the patient's oral health, and from that, the next step in or changes to a treatment plan.

Videos provided by the patient and/or generated from the reconstructed representation may also be used as inputs for purposes of training the model or as inputs to a trained model that operates as a classifier to identify an aspect of a patient's mouth, including their jaw and teeth. Such a model may be used for a diagnostic, monitoring, or visualization function based on intra-oral video, where such models benefit from a consistent, high-quality data source.

In one embodiment, an example of the disclosed method may include the following steps, stages, functions, or operations:

Acquire Image(s) or Video Using Mobile Device. This may be carried out by the patient using their mobile phone camera. In one embodiment, the desired timing (such as time of acquisition, or time interval between captured images), location, environmental conditions (lighting, temperature), camera settings, and/or other characteristics of the captured images or video may be determined and stored by an application or instruction set installed on the patient's device. In one embodiment, a user interface or audio segment may assist the patient in positioning the camera to capture a set of desired images (e.g., photos, video) (such as by providing guidance on the proper positioning of the camera for purposes of acquiring a desired image or video, such as how or where to move the camera, such as in translation or rotation). In one embodiment, an installed application on the mobile device may control the operation of the camera to assist in the capture of one or images at specific camera settings (such as by setting focus depth (or focus distance), aperture, focal length or illumination, as non-limiting examples). In one embodiment the mobile device's orientation or other characteristic relevant to an image or video may be determined and provided to the dentist or orthodontist along with the images or video (such as in the form of meta-data and/or sensor data). In one embodiment, sensor data (such as from a light sensor or gyroscope in the mobile device) may be used to provide guidance to the patient in positioning the mobile device or altering the environment conditions in which the images are collected. In one embodiment, the sensor data may be used during the processing of the images to correct them or modify them to remove artifacts or other undesired aspects.

The video and/or images are provided to the patient's dentist or orthodontist, either directly or via their accessing a remote platform (such as a multi-tenant or SaaS platform). In the situation of using as remote platform, each dentist or orthodontist may be associated with an account on a SaaS platform. Such a SaaS platform may include one or more services or applications for the processing and/or evaluation of the received images or video. In one embodiment, certain of the image processing functions may be performed on the mobile device. In one embodiment, the processing of captured images or video could include some form of anonymization, encoding, or encryption prior to transfer to a remote platform or system. If the patient captures a video, that video may later be processed to generate one or more images. This could include sampling or filtering, as non-limiting examples.

The images (e.g., photos, video) may be evaluated or otherwise filtered to obtain a set of desired images set for use in a reconstruction process. As is disclosed and/or described further herein, this stage of processing may involve one or more of the following for purposes of assessing (and in some cases, correcting) image quality (still image, video frame, or video frames). Detection of poor or insufficient image quality (such as by determining a degree of blur in an image or frame of video, or whether an initial image may not be correctable). Performing a comparison to previously obtained images to assist in identifying changes in tooth positions or alignment (or misalignment) between captures, and/or assist in evaluating the quality of an image provided by a patient. In one embodiment, this may be implemented as a form of supervised learning in which a trained model generates an indication of the relative quality or utility of an image (as a non-limiting example). In one embodiment, a dataset for use in training an image quality assessment model may be obtained by comparing labels or annotations assigned to images by multiple processes or techniques to identify labeling errors and using that information as part of the training data for a model.

The filtered or otherwise evaluated images (e.g., photos, video) may then be used as inputs to one or more reconstruction processes to generate a baseline representation for synthesis of a novel view or views as still images or moving video. Examples of image processing techniques that may be used to generate the reconstructed representation (or fusion) include, but are not limited to: differential volumetric rendering, such as Neural Radiance Fields (NeRFs), Structure from Motion, and Gaussian Splatting, Smart Image stitching, or Fine-tuned “personalized” diffusion model.

The reconstructed representation is then used to generate one or more images or videos that represent the patient's teeth, jaw or other aspects of the intraoral cavity as viewed from a desired perspective, location, environmental condition, or other aspect of interest to the dentist or orthodontist. This can enable the dentist or orthodontist to view the patient's teeth from other perspectives than those in the images (e.g., photos, video) obtained by the patient. This can also provide a baseline images and/or video for use by the dentist or orthodontist when comparing images or video data obtained at different times.

Evaluate the generated view or views to determine the current state of patient's teeth and progress of treatment as compared to a treatment plan. This process step may be used to monitor the progress of a treatment plan. The trained model may be used to create visual representations, including novel views, of a patient's dental anatomy, such the patient's teeth, from one or more static or dynamic views. The visual representations of the current state of the teeth may be evaluated to determine whether treatment is progressing as planned, such as by comparison to a model of the patient's detention in a treatment plan. In some embodiments, the visual representations of the current state of the teeth may be evaluated to make a diagnosis.

A machine learning model may be trained to act as a classifier using multiple generated images (e.g., photos, video). This is an optional stage but involves use of multiple generated images to train a model as a classifier for use in diagnosis, development of a treatment plan, or when trained, determination of the state or change in state of a patient's teeth. In one embodiment, the images used as part of a set of training data may be obtained from one or more of the following sources. The reconstructed representation disclosed and/or described herein, when operated under multiple conditions or input parameters. A set of images (e.g., a set of photos, set of videos) generated by a successive blurring process—in this approach, it is beneficial to determine the change in degree of blurring between images after successive application of a blur filter-if the change indicates that the original image was sufficiently clear, then the original and one or more of the blurred images may be used as part of a set of training data for a machine learning model.

A trained model may be operated to perform diagnostic, evaluation, or other functions, such as for aiding in generating an updated treatment plan and associated dental appliances, such as orthodontic aligners.

The trained model may be used to aid in dental visualization, diagnostics, monitoring, and other dental functions. For example, the model may be used to create visual representations, including novel views, of a patient's dental anatomy such the patient's teeth from one or more static or dynamic views. The model of a dental professional may monitor the changes in the patient's gingiva from a same perspective, such as a novel view, over time and based on, for example, changes in gingival recession, may make a diagnosis. A dental professional may monitor the changes in the patient's bite, chewing motion, or jaw articulation from a same novel view perspective over time and based on, for example, differing jaw movements and/or initial occlusal contacts, may make a diagnosis. Treatment plans, including updated dental appliances may be fabricated based on the diagnosis or evaluation.

In one embodiment, the disclosure is directed to a system for capturing intra-oral images (e.g., photos, video) of a patient, processing the captured images and/or to generate a reliable and consistent image or set of images (e.g., set of photos, set of videos) of the patient's teeth, jaw, and gums, and using the generated images or set of images to evaluate the current state of the patient's tooth positions, jaw articulation, bite, chewing motion or other aspect of the oral cavity. The system may include a set of computer-executable instructions stored in (or on) a memory or data storage element (such as a non-transitory computer-readable medium) and one or more electronic processors or co-processors. When executed by the processors or co-processors, the instructions cause the processors or co-processors (or a device of which they are part) to perform a set of operations that implement an embodiment of the disclosed method or methods.

In one embodiment, the disclosure is directed to a non-transitory computer readable medium containing a set of computer-executable instructions, wherein when the set of instructions are executed by one or more electronic processors or co-processors, the processors or co-processors (or a device of which they are part) perform a set of operations that implement an embodiment of the disclosed method or methods.

In some embodiments, the systems and methods disclosed herein may provide services through a SaaS or multi-tenant platform. The platform provides access to multiple entities, each with a separate account and associated data storage. Each account may correspond to a dentist or orthodontist, a patient, an entity, a set or category of entities, a set or category of patients, an insurance company, or an organization, for example. Each account may access one or more services, a set of which are instantiated in their account, and which implement one or more of the methods or functions disclosed and/or described herein.

Other objects and advantages of the systems, apparatuses, and methods disclosed will be apparent to one of ordinary skill in the art upon review of the detailed description and the included figures. Throughout the drawings, identical reference characters and descriptions indicate similar, but not necessarily identical, elements. While the embodiments disclosed or described herein are susceptible to various modifications and alternative forms, specific embodiments are shown by way of example in the drawings and are described in detail herein. However, embodiments of the disclosure are not limited to the exemplary or specific forms described. Rather, the disclosure covers all modifications, equivalents, and alternatives falling within the scope of the appended claims.

The terms “invention,” “the invention,” “this invention,” “the present invention,” “the present disclosure,” or “the disclosure” as used herein are intended to refer broadly to all the subject matter disclosed in this document, the drawings or figures, and to the claims. Statements containing these terms do not limit the subject matter disclosed or the meaning or scope of the claims. Embodiments covered by this disclosure are defined by the claims and not by this summary. This summary is a high-level overview of various aspects of the disclosure and introduces some of the concepts that are further described in the Detailed Description section below. This summary is not intended to identify key, essential or required features of the claimed subject matter, nor is it intended to be used in isolation to determine the scope of the claimed subject matter. The subject matter should be understood by reference to appropriate portions of the entire specification, to any or all figures or drawings, and to each claim.

### BRIEF DESCRIPTION OF THE DRAWINGS

Embodiments of the disclosure are described with reference to the drawings, in which:

FIG. 1(a) is a flowchart or flow diagram illustrating a process, method, operation, or function that may be performed in an implementation of an embodiment of the disclosed system and methods;

FIG. 1(b) is a flowchart or flow diagram illustrating a process, method, operation, or function that may be performed for evaluation of the progress of a treatment plan in an implementation of an embodiment of the disclosed system and methods;

FIG. 1(c) is a flowchart or flow diagram illustrating a process, method, operation, or function that may be performed for Image Capture or Image Quality Assessment in an implementation of an embodiment of the disclosed system and methods;

FIG. 1(d) is a view of a user interface for displaying generated and original images;

FIG. 1(e) is a flowchart or flow diagram illustrating a process, method, operation, or function that may be performed for Blurriness Image Quality Assessment in an implementation of an embodiment of the disclosed system and methods;

FIG. 1(f) is a flowchart or flow diagram illustrating a process, method, operation, or function that may be performed for generating novel views using a differentiable volumetric rendering model in an implementation of an embodiment of the disclosed system and methods;

FIG. 1(g) is a flowchart or flow diagram illustrating a process, method, operation, or function that may be performed for generating and manipulating a morphable 3D model in an implementation of an embodiment of the disclosed system and methods;

FIG. 2 is a diagram illustrating elements or components that may be present in a computer device or system configured to implement a method, process, function, or operation in accordance with some embodiments of the systems, apparatuses, and methods disclosed herein; and

FIGS. 3-5 are diagrams illustrating an architecture for a multi-tenant or SaaS platform that may be used in implementing an embodiment of the systems and methods disclosed herein.

Note that the same numbers are used throughout the disclosure and figures to reference like components and features.

### DETAILED DESCRIPTION

Capturing high quality and consistent image data, such as still images or video that is suitable for clinical evaluation and dental treatment planning over time presents many challenges due to variations in lighting, the capture equipment used, changes in dental conditions and other factors. These challenges may be increased when a patient captures and provides the image data. Discussed herein, we provide solutions and improvements to the capture and processing of digital image data that provides many advantages. For example, digital images data may be captured at different orientations, positions, focal lengths, and at different focus distances or depths. The digital image data may even be captured at inconsistent orientations, positions, focal lengths, and depths of focus. A full dental model may be generated from the digital image data. The image data, or portions thereof, may be assessed for quality and assigned a quality metric. Image data may be selected for use in generating the full dental model, articulation model, or other model of the oral cavity based on their quality, including focus, appropriate exposure, or other factors, as discussed herein.

The systems and methods discussed herein aid in providing more consistent progress tracking in both 2D and 3D image spaces of both static and dynamic (e.g., moving) subjects, treatment planning, and other dental applications. They improve the operation of dental computing systems and processes by aiding in generating accurate and high quality data that would not otherwise be available.

In some embodiments, the systems, apparatuses, and methods disclosed and/or described herein are directed to systems, methods, and apparatuses for enabling a patient to capture intra-oral images (e.g., photos, video) and for the processing of those images to assist a dentist, orthodontist, or other dental professions in evaluating the state of a patient's oral health, including teeth, jaw, etc. and the development or progress of a treatment plan.

For example, in some embodiments, the captured images (e.g., photos, video) are used as the basis for developing a reconstructed representation of the current state of a patient's teeth by “fusing” information obtained from multiple images. The reconstruction can be used to generate novel images of a specified area of the mouth and/or images taken from a desired perspective, patient movement, or environmental condition. This enables a dentist to better determine the current state of the patient's dentition and to evaluate and/or refine a treatment plan. The described and/or disclosed approach also provides a capability for a dentist or orthodontist to have a consistent view of the patient's dentition when monitoring a treatment plan over time.

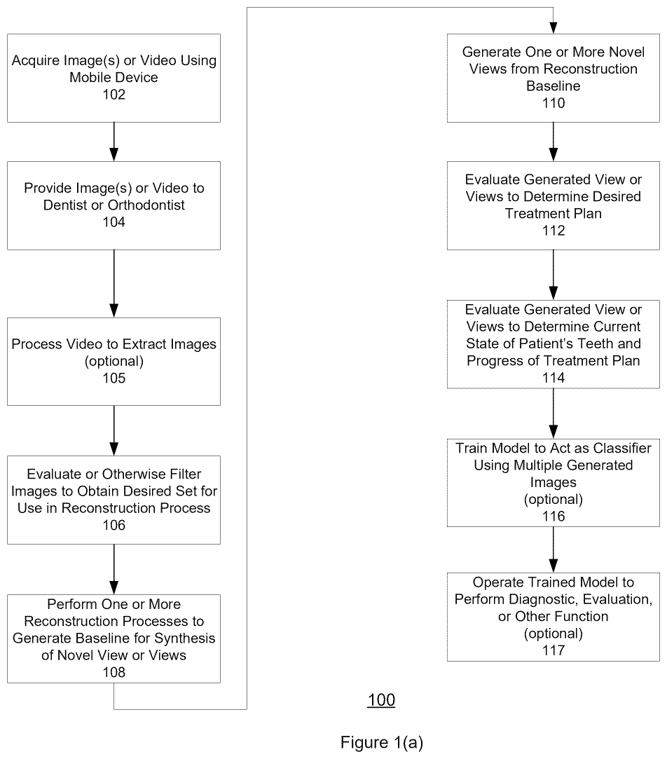

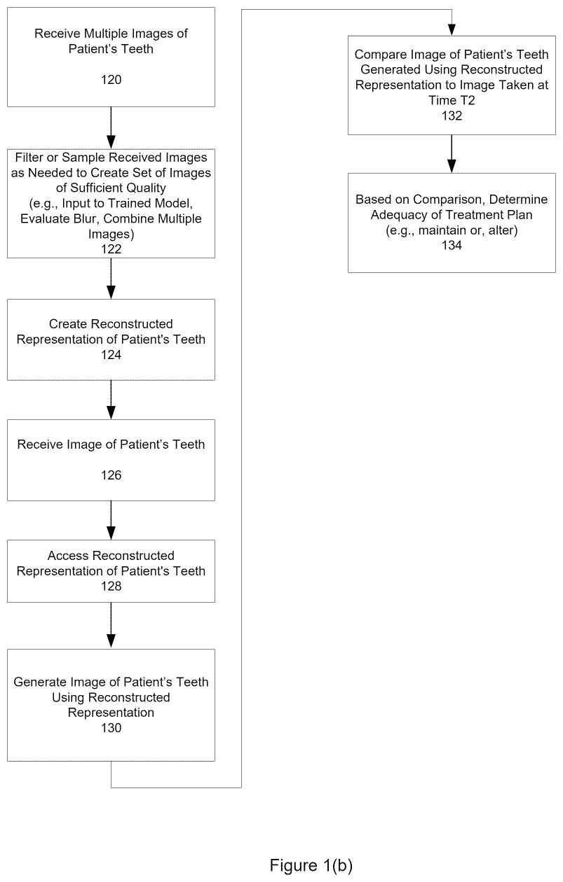

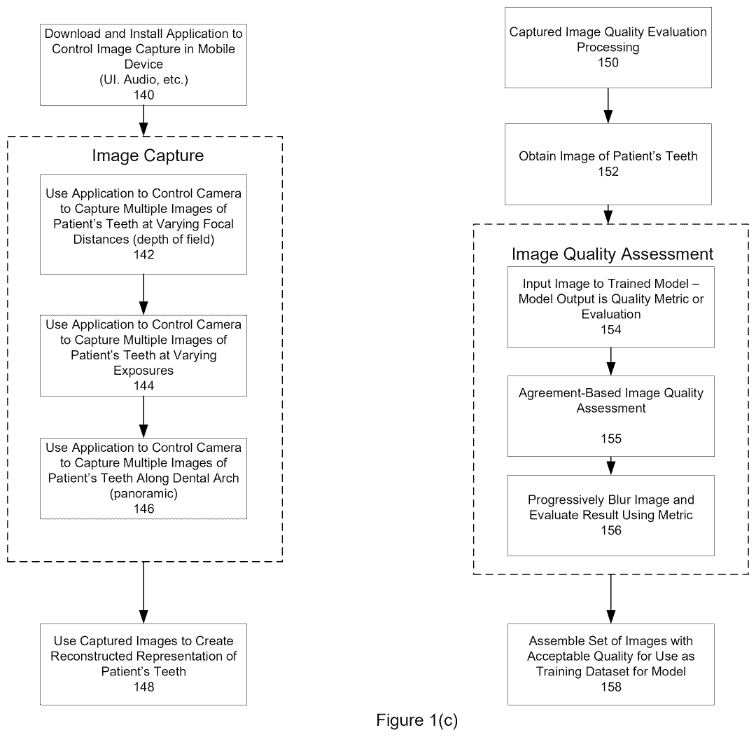

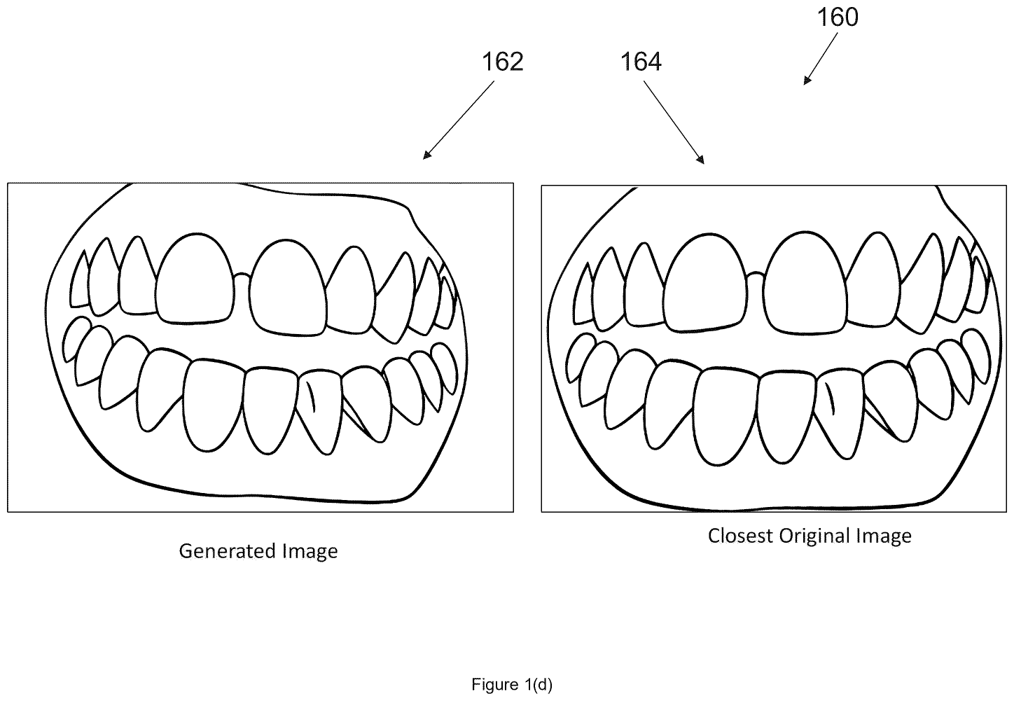

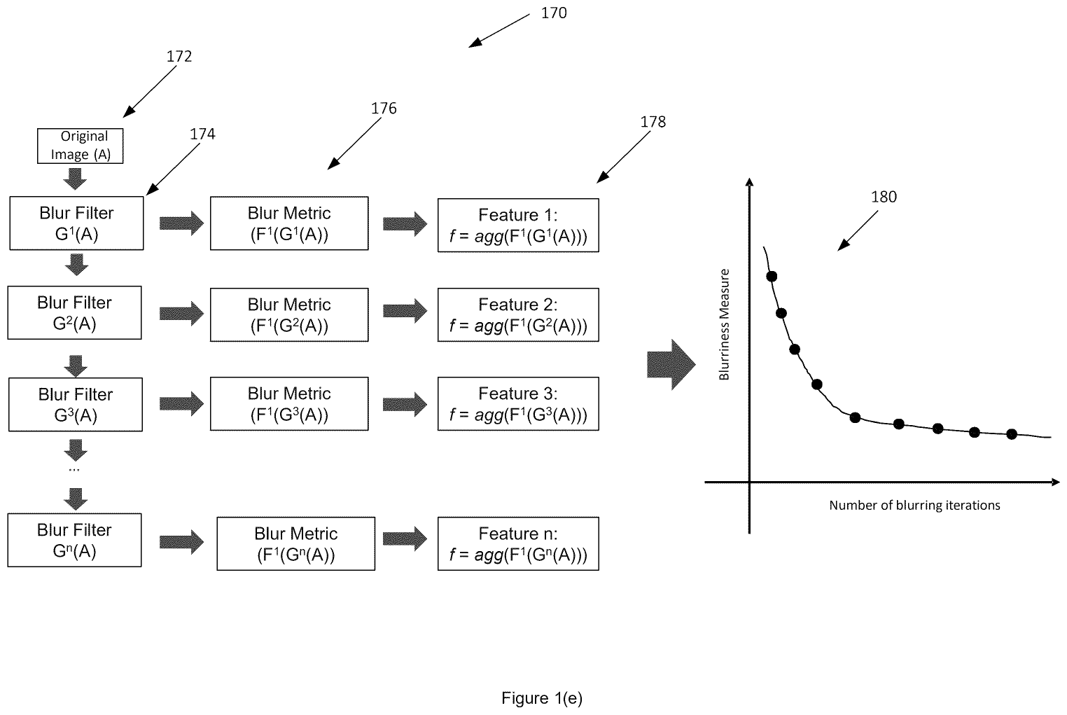

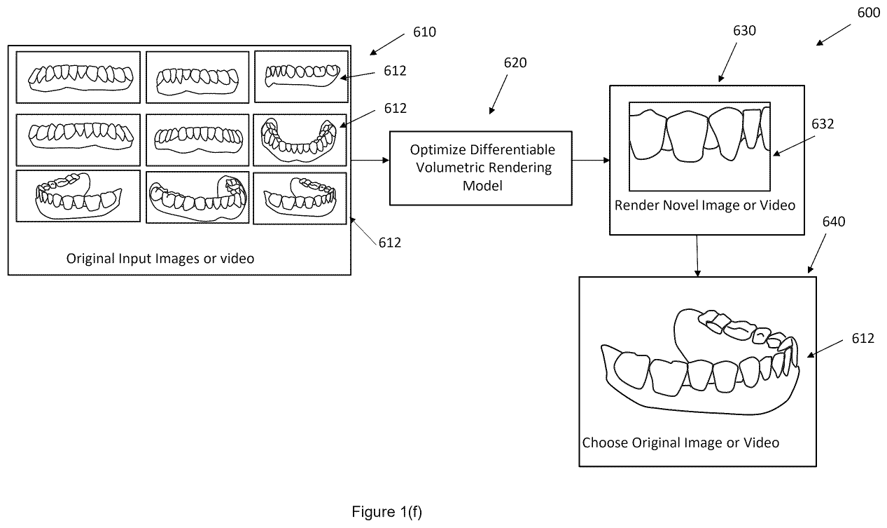

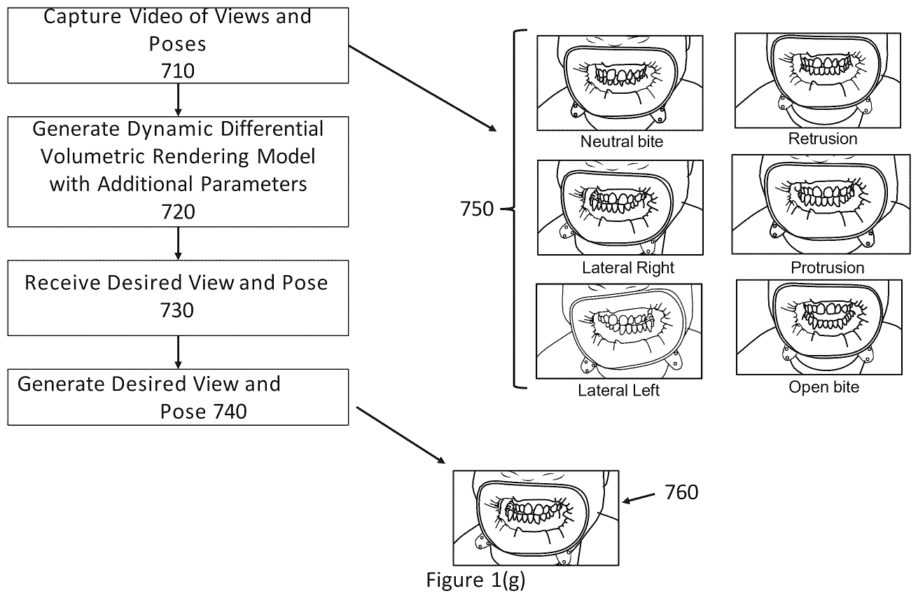

FIG. 1(a) is a diagram illustrating a set of processes, methods, operations, or functions 100 that may be performed in an implementation of an embodiment of the disclosed system and methods. As shown in the figure, an embodiment may comprise one or more of the following steps or stages.

At block 102, images or video of a patient's intraoral cavity, which may include hard tissue, such as teeth, soft tissue, such as the gingiva, lips, and/or check is acquired using a mobile device. In some embodiments, the images or video may include extraoral features or anatomy, such as the those of the face, including the nose, eyes, etc. In one embodiment, the desired timing (such as time of acquisition, or time interval between captured images), location, environmental conditions (lighting, temperature), camera settings, and/or other characteristics of the captured images or video may be determined and stored by an application or instruction set installed on the patient's device. In some embodiments, images may include 2D images, such as digital images captured using image sensors, such as a CMOS or CCD image sensors, or image data, such as digital data that describes the light captured by an image sensor. The light may be visible light reflected from the surfaces of the patient's tissue, or reflected from subsurface tissue, such as IR light that may penetrate the patient's tissue. In some embodiments, the light may be x-ray or other wavelengths of light and the image data may record the light the passes through the patient's tissue. In some embodiments, images may include 3D image data, such as a point cloud representing surface data of the patient's dentition, a 3D mesh representing surface data. In some embodiments, the image data may include, 3D volumetric data, such as CBCT data, CT scan data, or other volumetric data. Two-dimensional data may be represented by a 2D arrangement (such as in a 2D array) of pixels. Three-dimensional data may be represented by a 2D or 3D arrangement of voxels (such as a 3D array).

In one embodiment, a user interface or audio segment may assist the patient in positioning the camera to capture a set of desired images, such as by providing guidance on the proper positioning of the camera for purposes of acquiring a desired image or video. For example, if the camera is too close, the guidance may include direction to move the camera further from the patient's dentition. If the camera is tilted, the guidance may include direction to rotate the camera. If the camera is moving too fast or too slow, the guidance may include direction to slow down, hold the camera still, or to move the camera to along the arch of the patient's dentition.

In one embodiment, a user interface or audio segment may assist the patient in moving the camera or aspects of their intraoral cavity, such as their jaw, lips, etc. to capture a set of desired images (e.g., photos, video) of dynamic patient movement during the movement (such as by providing guidance on the proper camera or anatomical positioning for purposes of acquiring a desired image or video, such as how or where to move the camera, jaw, lips, etc.).

In one embodiment, an installed application on the mobile device may control the operation of the camera to assist in the capture of one or images (e.g., photos, video) at specific camera settings, such as by setting focus distance, focal length or illumination, such as by automatically turning on a light, as non-limiting examples.

In one embodiment the mobile device's position and orientation, such as relative to the patient's dentition, or other characteristic relevant to an image or video may be determined and provided to the dentist or orthodontist along with the images or video (such as in the form of meta-data and/or sensor data). In one embodiment, sensor data (such as from a light sensor or gyroscope in the mobile device) may be used to provide guidance to the patient in positioning the mobile device or altering the environment conditions in which the images are collected, such as to provide guidance, as discussed herein. In one embodiment, the sensor data may be used during the processing of the images to correct them or modify them to remove artifacts or other undesired aspects.

At block 104, images or video may be provided to a dentist or orthodontist, for evaluation. For example, the mobile device may send the images or video (and related information) to a remote server that may store the images or video and enable the dentist or orthodontist to then access the images or video (and related information). As another example, the mobile device may send the images directly to a system of the dentist or orthodontist.

In some embodiments, if the patient captures a video, that video may later be processed to generate one or more images. For example, at block 105, the video may be processed to extract images. Processing may also include sampling or filtering, as non-limiting examples.

At block 106, the images (e.g., photos, video) may be evaluated or otherwise filtered to obtain a desired set of images for use in a reconstruction process, such as a reconstruction of a novel view of the patient's dentition. As is disclosed and/or described further herein, this stage of processing may involve assessing (and in some cases, correcting) image quality. Assessing or correcting image quality may include detection of poor or insufficient image quality (such as by determining a degree of blur in an image, or whether an initial image may not be correctable), performing a comparison to previously obtained images to assist in identifying changes in bite, chewing motion, jaw articulation, tooth positions or alignment (or misalignment) between pictures, and/or assist in evaluating the quality of an image provided by a patient.

In one embodiment, assessing or correcting image quality may be implemented as a form of supervised learning in which a trained model generates an indication of the relative quality or utility of an image (e.g., photos, video) (as a non-limiting example). In one embodiment, a dataset for use in training an image quality assessment model may be obtained by comparing labels or annotations assigned to image data, such as images or video, by multiple processes or techniques to identify labeling errors and using that information as part of the training data for a model.

At block 108, the filtered or otherwise evaluated images (e.g., photos, video) are used as inputs to one or more reconstruction processes to generate a baseline for synthesis of a novel static or dynamic view or views of the patient's dentition.

Examples of image processing techniques that may be used to generate the reconstruction representation (or fusion) include, but are not limited to differential volumetric rendering techniques such as, Neural Radiance Fields and Gaussian Splatting, image stitching, structure from motion, and fine-tuned personalized diffusion models. The use of such image processing techniques are described in further detail below.

At block 110, one or more novel static or dynamic views are generated from the reconstructed representation of the patient's teeth or a baseline representation of the patient's teeth. The reconstructed representation is then used to generate one or more images or videos that represent the patient's oral cavity as viewed from a desired perspective, location, environmental condition, movement, facial or jaw position, or other aspect of interest to the dentist or orthodontist. This can enable the dentist or orthodontist to view the patient's oral cavity from other perspectives and in positions other than those in the images or video obtained by the patient. This can also provide a baseline image or images (e.g., photos, videos) for use by the dentist or orthodontist when comparing images obtained at different times.

At block 112, the generated static or dynamic view or views are evaluated to determine a desired treatment plan. This process step may be used for the preparation of an initial treatment plan. A treatment plan may include one or more treatment stages. The treatment stages may be incremental repositioning stages of an orthodontic treatment to move one or more of the patient's teeth from an initial tooth arrangement towards a target arrangement. For example, the treatment stages can be generated by determining the initial tooth arrangement indicated by the digital representation, such as from the generated view or views, determining a target tooth arrangement, and determining movement paths of one or more teeth in the initial arrangement to achieve the target tooth arrangement. The movement path can be optimized based on minimizing the total distance moved, preventing collisions between teeth, avoiding tooth movements that are more difficult to achieve, or other suitable criteria. The treatment stages may be incremental jaw repositioning stages of an orthodontic treatment to move the patient's jaws from an initial arrangement towards a target arrangement, such as through mandibular advancement or retraction. The treatment plan may also include restorative treatment, such as a veneer, crown, bridge, or other restorative treatment.

At block 113, the generated static or dynamic view or views are evaluated to determine the current state of the patient's teeth and progress as compared to a treatment plan. This process step may be used to monitor the progress of a treatment plan. Progress tracking may occur at any point during treatment. In some embodiments, progress tracking may correspond with a patient completing a pre-planned phase of treatment, such as fitting of a crown or other prosthetic, completing a orthodontic treatment stage, which may include the wearing of an orthodontic aligner for a period of time. For example, once a treatment plan has been determined, a first set of one or more appliances to be administered to the patient in a first phase of treatment, which may include one or more stages. Progress tracking may occur after the last appliance in the first set is administered to the patient. In some embodiments, progress tracking may occur after an indication that the patient's treatment progress is not tracking with the expected or planned progress, such as if an aligner for a stage of treatment does not fit on the patient's arch. For example, the aligner may be retained on the arch so well that it is difficult for the patient to remove the aligner or the aligner may be retained so little that the aligner of a portion therefore disengages from the arch.

At block 116, a machine learning model may be trained to act as a classifier using multiple generated images (e.g., photos, video). This may include use of multiple generated images to train a model as a classifier for use in diagnosis, development of a treatment plan, or when trained, determination of the state or change in state of a patient's oral cavity. In one embodiment, the images used as part of a set of training data may be obtained from one or more sources. This may include use of multiple generated images to train a model as a classifier for use in diagnosis, development of a treatment plan, or when trained, determination of the state or change in state of a patient's oral cavity. In one embodiment, the images used as part of a set of training data may be obtained from one or more of the following sources: the reconstructed representation disclosed and/or described herein, when operated under multiple conditions or input parameters, a set of existing images and annotations that have been evaluated to determine their labeling accuracy, and/or a set of images generated by a successive blurring process—in this approach, it is beneficial to determine the change in degree of blurring between images after successive application of a blur filter-if the change indicates that the original image was sufficiently clear, then the original and one or more of the blurred images may be used as part of a set of training data for a machine learning model.

At block 117, the trained model may be used to aid in dental visualization, diagnostics, monitoring, and other dental functions. For example, as discussed herein, the model may be used to create visual representations of a patient's dental anatomy, such as the current state of the patient's teeth from one or more dynamic or static views. In some embodiments, the images may be used in aiding diagnostic applications. Dental diagnostics includes identifying and aiding in treating various oral health issues including diagnosing health issues with teeth, gums, and other structures in the mouth. Dental monitoring may include tracking the progress and status of a patient's dental health and orthodontic treatment over time. For example, the trained model may be used to generate multiple static or dynamic visualizations of the patient's dentition from the same perspective (including field of view and focal length) over time, such as based on multiple imaging sessions spaced out over a period of time. A dental professional may monitor the changes in the patient's gingiva from that same perspective over time and based on, for example, changes in gingival recession, may make a diagnosis. A dental professional may monitor the changes in the patient's bite, chewing motion, or jaw articulation from that same perspective over time and based on, for example, differing jaw movements and/or initial occlusal contacts, may make a diagnosis.

As described, one or more reconstruction processes may be used to “fuse” multiple images (e.g., photos, videos) and generate a baseline for the synthesis of a novel static or dynamic view or views of the patient's oral cavity, including jaw movements, teeth, and gums. There are several such processes that are available and may be utilized. For example, differential volumetric rendering techniques, such as neural radiance fields and Gaussian Splatting, smart image stitching, and fine-tuned personalized diffusion modeling.

In some embodiments, the images or video of the patient's dentition may be used to generate a neural radiance field of the patient's dentition. A neural radiance field is a method for synthesizing realistic 3D scenes from a set of 2D images (e.g., photos, videos). A neural radiance field may represent a scene as a continuous function, such as a 5-dimensional function (x, y, z, coordinates that define a location in the scene and two angles that define a viewing direction) that maps a 3D coordinate and a 2D viewing direction to a color and density at that point. This approach allows for producing photo-realistic renderings of still or dynamic subjects from novel viewpoints (viewpoints not captured in the 2D images used to generate the neural radiance field).

In some embodiments, a neural radiance field represents a scene, such as the dentition, including the teeth and gums of the patient, using a neural network. The neural network is trained on the images and/or video of the patient's dentition, such as those acquired at block 102. During training, the network learns to predict the color and density of points in the scene such that, when these points are rendered from the same viewpoints as the training images or video, they closely match the original images. This process may include optimizing the parameters of the network using gradient descent. In some embodiments, additional images, videos, or other data may be used to train the neural network. For example, the position and orientation data captured when the images or video were captured may also be used to train the network. In some embodiments, scan data or 3D models of the patient's teeth or generic teeth may be used to train the model. Such dentition specific data and/or position and orientation data may aid in a more accurate neural network and speed up the generation of the neural network.

After training the neural network, the neural network may be used to generate an image (e.g., photos, video) from a novel viewpoint. The image may be generated using volume rendering. When using a volume rendering technique, rays are cast from the novel viewpoint into the scene. Points along each ray are sampled for color (such as in every voxel of a 3D scene). Using the neural network each pixel is generated (such as by assigning color properties for the pixel) based on sampled rays, for each point or pixel in the generated 2D image or frame of video.

As discussed herein, a neural radiance field can be used to effectively fill in gaps in the original image data captured during the progress tracking image acquisition at block 102. As discussed herein, a patient or even a dentist may not capture progress images (e.g., photos, video) from the same camera position and orientation or the same subject movements during each stage of progress tracking. A neural radiance field may be used to allow for the generation of consistent images from the same perspective over time, even when the original acquired images or videos is not captured from the same perspective. This capability is particularly valuable for intra-oral imaging, as it can rectify patient capture errors and produce images that align seamlessly with a consistent perspective and may also correct for patients' image capturing errors.

Static neural radiance fields may generate novel views of a static scene captured from multiple viewpoints. These fields are particularly useful in reconstructing a scene that remains unchanged over the imaging period. By leveraging the continuity and consistency of static scenes, static NeRFs achieve high levels of detail and accuracy in the produced images. In some embodiments, dynamic neural radiance fields may be used to expand the capabilities of static NeRFs by incorporating time as an additional input variable, enabling the rendering of time-varying 3D scenes with deformations (rigid or even non-rigid), e.g., from sparse monocular or multi-ocular input views. These dynamic neural radiance fields may be used in generating novel views, including frames of videos, from a dynamic scene in which the subject, the scene, or elements within the scene move during capture. The dynamic neural radiance fields may generate video (or still images) of the subject's movement from novel views. Dynamic neural radiance fields extend to dynamic scenes by incorporating time as an additional input variable. This enables rendering time-varying 3D scenes with non-rigid deformations from sparse monocular or multi-ocular input views.

A dynamic neural radiance field decomposes the learning process into two neural networks, a canonical network and a deformation network. The canonical network is trained to learn the scene's appearance and geometry in a canonical (e.g., reference) configuration and the deformation network is trained to model the transformation between the dynamic scene at various times and the canonical space. This method may allow dynamic NeRFs to generate temporally coherent renderings of novel views, accommodating the changes in the scene over time. The dynamic neural radiance field model is trained to learn how each point in a scene moves over time. Given a desired novel view or views, the dynamic neural radiance model uses the canonical network and deformation network to generate a temporally coherent rendering of novel views.

With monocular or multi-ocular views, the deformation field learning may map each point at a particular time to its corresponding canonical space representation using a deformation network, which is used to predict one or more displacement vectors for the point. The canonical network models the radiance and density of the dynamic scene in a fixed (e.g., canonical) space to yield a color and a volume density for points within the canonical space.

During the rendering process for a novel view at a particular time the deformation network may map each point into the canonical space. The canonical network may then generate color and density values for the mapped point and, finally, volume rendering is used to synthesize the final image or images for a video.

In some embodiments, depth information may be generated from the input video and used with the differential volumetric rendering described herein. Depth information may be generated from a monocular video based on a deformation field trained to map points from different video frames into canonical space. During training, an initial depth estimation is made for each frame in a video. Then a depth scale is applied to correct the depth scale differences by segmenting static and dynamic regions in the video, reprojecting depth values across the frames into a common reference frame, and determining the scale factor for reach location for each frame correlate the depth values across the frames. The deformation field is then generated based on depth scales across multiple frames.

Dynamic Neural Radiance Fields may aid in dental treatment in many ways. For example, Dynamic Neural Radiance Fields may aid in reconstructing and analyzing 3D jaw movements from captured video data, allowing clinicians to generate novel viewpoints and simulate orthodontic treatment, including aesthetic modifications. This capability may be used in remote dental assessments, treatment planning, and patient education, where capturing clinically preferred perspectives by patients recording their own jaw movement videos may be challenging.

One of the challenges in dental evaluations is the inconsistency of camera angles when patients record jaw movement videos using personal devices. Even recordings by dental professionals may not be consistent or captured with clinically preferred viewpoints. These recordings may vary in distance, angle, and stability, leading to suboptimal views for clinical analysis. Dynamic NeRFs may address this issue by reconstructing a videos of the patient's jaw captured at different angles, distances, etc., at a consistent and novel viewpoint. This allows the system to render novel videos from a clinically preferred viewpoint, such as frontal occlusal or left or right side views, providing dental professionals with an optimal perspective for assessing jaw alignment, occlusion, Temporomandibular joint function, and other dental assessments without the patient re-recording videos, recording videos under clinical supervision, or an in-personal visit.

In addition to capturing a single instance of jaw motion, clinicians may monitor a patient's jaw function over weeks or months to track treatment progress. However, videos captured at different times may have inconsistencies in camera positioning, lighting, and distance, making direct comparisons difficult. Dynamic NeRFs may overcome these limitations by normalizing each recording, through novel view generation, to a standardized clinical perspective, allowing for consistency in visualizing jaw motion over time. This allows dental professionals to accurately compare pre-treatment, mid-treatment, and post-treatment changes, to detect deviations in mandibular movement, and assess progress in orthodontic, prosthetic, or jaw occlusion treatments.

In some embodiments, dynamic NeRFs may be used in evaluating modifications to a patient's dentition. Dynamic NeRFs may allow clinicians to generate synthetic images of a modified teeth arrangement, such as with orthodontic adjustments, veneers, or prosthetic reconstructions, and then use these images to render realistic motion videos. Dental professionals may use the novel view generation with the modified teeth or detention to evaluate functional adaptations, showing how different dental treatments might impact a patient's bite and jaw movement over time. Patients may use them to better visualize the potential outcomes of treatment, helping them make informed decisions about their dental care.

For at least some clinical evaluations or applications, an evaluation of the real-world images (e.g., photos, video) (as opposed to synthesized images) may be used. With reference to FIG. 1(f) a method 600 is shown. The method 600 may start at block 610 where a plurality of images 612 of the patient's dentition are captured from one or more of multiple viewpoints, focal lengths, depths of focus, etc. At block 620 a NeRF model is optimized and/or trained based on the input images data (e.g., photos, video) and/or other data. At block 630, a dental professional may review the novel view 632 of the static or dynamic subject synthesized by the neural radiance field to monitor the patient's dentition. In some embodiments, at block 640, one or more original images or videos 612 that were captured from one or more perspectives that are closest to the novel view, may be presented to the dental professional for review (e.g., if a location on the dentition or jaw movement, etc. is concerning or otherwise warrants further review). For example, the system disclosed herein may present a synthesized novel view of the static or dynamic subject and may also identify a set of original images that depict a scene of the novel view in a perspective that is within a threshold degree of similarity (e.g., in terms of the angle and coordinates of features within the scene). Additionally, the uncertainty associated with a novel view may be provided to the dental professional to allow them to determine whether or not they should view the originally captured data of a particular location on the patient's dentition. may be provided to the dental professional to allow them to determine whether or not they should view the originally captured data of a particular location on the patient's dentition.

FIG. 1(d) depicts a user interface 160, such as shown on a display, of a novel view 162 of a static or dynamic subject based on a reconstructed representation, such as generated using neural radiance field (or the other reconstruction representation methods described herein, such as structure from motion, Gaussian Splatting, and others) next to an original image 164 that was captured from the closest position and orientation as depicted in the novel view 162.

In some embodiments, a 3D model of the patient's dentition may be generated using the neural radiance field. For example, multiple novel views may be generated from the neural radiance field, these views may be used to generate a 3D model using photogrammetry, structure from motion (SfM), or other methods.

In some embodiments, Gaussian Splatting may be used to generate novel static or dynamic views of the patient's dentition. Gaussian Splatting may generate results similar to those of NeRFs but with some differences and advantages. Unlike NeRFs, there is no neural network component required for Gaussian Splatting, and rendering using Gaussian Splatting may be faster. Gaussian Splats are a collection of points in the space of a scene represented as 3D Gaussian distributions with position, rotation, and scale (each having a mean and a standard deviation). Each point may have a “color” represented by 3rd order Spherical Harmonic coefficients, allowing the color to change based on the view direction. During rendering into a novel 2D image or video (such as frames of a video), the points are represented as 2D Gaussians in the image space, such as a 2D array of pixels. This method handles specular highlights and reflections well, which are prevalent in intra-oral images due to saliva reflection, and may make it especially suitable for this use case.

Dynamic Gaussian Splatting may be used to render novel static or dynamic views of a dynamic scene. In a dynamic Gaussian Splatting model, each point in space in a scene has the same scale, color, and opacity over time, similar to non-dynamic Gaussian Splats. However, in dynamic Gaussian Splatting, each Gaussian also has a position and rotation in 3D space that is tracked and moves over time (such as in the time of the original captured video). Additionally, each Gaussian may have a background logit. A background logit isa logit, the value of which may be used to help the model differentiate between Gaussians that are part of the dynamic object or subject and Gaussians that are part of a static background within the captured video. A high value for a background logit may indicate that the Gaussian is part of the static background of a scene while a low or negative value may indicate that a Gaussian is part of the dynamic portion of a scene. In some embodiments, the value of the background logit is a probability that the Gaussian is part of the background. This means that while the visual characteristics of each point remain constant, their positions and orientations are updated frame by frame to reflect the movement within the scene. Essentially, dynamic Gaussian Splatting may allow for the creation of continuous and realistic representations of moving subjects or other elements in videos by combining static properties with dynamic positional data. Gaussian

When generating a dynamic Gaussian Splatting model, at a first point in time the Gaussian parameters are initialized from a sparse point cloud. Then in subsequent video frames, position and rotation parameters for the Gaussians are updated based on the changing scene while scale, opacity, and color remain fixed. By keeping scale, color, and opacity fixed over time, dynamic Gaussian Splatting enforces consistent color, size, and opacity during novel view generation. When rendering a novel view at a particular time using the dynamic Gaussian Splatting model Gaussian Splatting uses the parameters for that particular time. For example, a Gaussian Splat for a frame generated to represent a novel view of a scene at time T1 may be generated based on the position and rotation parameters at time T1 in the model while a Gaussian Splat for a frame generated to represent a novel view of a scene at time T2 may be generated based on the position and rotation parameters at time T2. Rendering a novel view or views of a dynamic scenes over a period of time results in a series of images that can be used to form a video.

The systems and methods herein describe the generation of novel static and dynamic views. While some embodiments describe the generation of novel views using Gaussian Splatting and others describe the generation of novel views using NeRFs, it is to be understood that where the use of Gaussian Splatting is discussed, NeRFs may also be used instead, and where the use of NeRFs is discussed, Gaussian Splatting may also be used instead.

In some embodiments, generating a novel view using Gaussian Splatting may include capturing a scene, such as through capturing 2D images, video, etc. For example, a mobile device (e.g., a smart phone or other camera device) may be used to capture such images or video. In some embodiments, 3D image data may also be generated. For example, 3D scanning, photogrammetry, or depth sensing may be used during scene capture. Key features or points may be extracted from the scene. These features may be 3D points in space and include position, color values, texture information, etc. Each of these features may be represented with a Gaussian distribution for position, color values, texture information, etc. The parameters of the Gaussian (the mean and distribution) can be determined based on the desired level of smoothness or blur.

When generating a novel view (i.e., an image or video from a viewpoint not originally captured), the system calculates the contribution of each feature to each pixel in the novel view. This may be performed by projecting the Gaussian distributions of the features onto the new view plane. Contributions from different features are accumulated at each pixel. The Gaussian Splatting allows features to smoothly blend into each other, which aids in avoiding discontinuities. The accumulated values at each pixel are then used to generate the final image or frame of video, providing a realistic and continuous representation of the static or dynamic scene from the new viewpoint.

Novel views may be used by dental professionals, for example, to assess progress of a dental treatment. For example, novel views may allow for more direct comparisons between images (e.g., photos, video) taken at two different times (which may vary in angle/position), as discussed further herein (e.g., with respect to FIG. 1(b)). As another example, a dental professionally may be able to generate a desired view that may not have been captured to better assess a region (e.g., a prominence of a tooth) or to generate a set of views around a region to gain a three-dimensional understanding of the region.

As noted above, in clinical evaluations or applications requiring evaluation of real-world images, a dental professional may first review a novel 2D image (e.g., photos, video) synthesized by Gaussian Splatting to monitor the patient's dentition, and may subsequently review (or request) one or more originally captured images that are closest to the synthesized novel view. If a location on the dentition is concerning or otherwise warrants further review, the original image or images (e.g., photos, videos) that are captured from the closest or a set of close perspective as compared to the novel view, may be presented to the dental professional for review. Additionally, the uncertainty associated with a novel view may be provided to the dental professional to allow them to determine whether or not they should view the originally captured data of a particular location on the patient's dentition.

In some embodiments, a 3D model of the patient's dentition may be generated using Gaussian Splatting. For example, multiple novel views may be generated from the Gaussian Splatting. These views may be used to generate a 3D model using photogrammetry, structure from motion, or other methods.

In some embodiments, image stitching may be used to generate novel images (e.g., still or video) with a greater field of view, such as a panoramic view. Image stitching may use one or more of a combination of different methods to generate novel images. Features in the initial captured images, either still images or video, are matched. Methods for this may include classic algorithms such as SIFT or machine learning based methods such as SuperGlue or LightGlue to match key points across frames. The images are then transformed and stitched together to create a panoramic view based on the key points. In some embodiments, color filters are applied to each transformed image to provide consistent coloring in the image. Color filters may be used to mitigate inconsistencies caused by differences in image capture conditions, such as due to images being captured under different lighting or exposure conditions.

In some embodiments, a fine-tuned personalized diffusion model may be used to generate novel views. The approach is to fine-tune a diffusion model using patients' intra-oral images and then generate novel viewpoints from this model. This approach uses a diffusion model that is trained in two parts. In a first part, a diffusion model may be trained based on images of a dentition that is not of the patient. For example, the images may be of a generic dentition with generic teeth, gingiva, and another tissue from multiple different viewpoints. In some embodiments, the model may be additionally or alternatively trained using 3D models of a generic dentition, such as 2D rendered views of the dentition from many viewpoints. The viewpoints and other aspects of the training data may be labeled to include information such as the location of the teeth, gingiva, and other tissue within the image and/or the distance and angles of the viewpoints. In a second part, which may occur after the first part, the model may then be trained using labeled images of the patient's actual dentition. After training the model with the first part and the second part, a highly specific image is synthesized in various scenarios, including different viewpoints.

Once the reconstruction process or processes are applied, the reconstructed representation may be used to generate one or more novel views of a patient's teeth, gums, lips, palate, etc. Parameters or a description of a desired view, such as “right buccal view,” may be specified by a user of the reconstruction process and used to cause the process to generate one or more images, such as a right buccal view of the patient's dentition from a particular angle, distance, and field of view. This will enable the dentist or orthodontist to better visualize the patient's mouth and teeth and provide a common view or perspective for purposes of comparison which may be lacking in the images provided by a patient at different times. Methods also exist to quantify how uncertain areas of a novel view are to assist the viewer in determining how much to rely on them.

In some embodiments, the structure of objects in a scene may be determined based on the relative motion of the objects when images from different locations or points of view, such as different angles and distances. A structure from motion model may be generated from a series of 2D images or video captured from different angles and positions around the dentition. The images include overlapping fields of view, capturing the portions of the same location of the dentition in multiple images from different angles and positions.

Key points in each image are then identified. The key points of an image may identify locations within the image that correspond to distinctive features, such as edges of the teeth and/or gingiva; colors; or marks on the surface of teeth and/or gingiva, tooth cusp, interproximal areas; etc. The key points and the features they correspond to may then be matched across the set of images, determining where features in one image are located in the other images in the set.

The camera's pose, such as the location and angle, when taking the image is then determined based on the relative locations of the key points in the images. In some embodiments, a 3D model of a dentition, such as a generic dentition or the patient's dentition may be used to aid in determining the camera pose when taking an image. For example, multiple 2D projections of the 3D model of the dentition may be generated at various virtual camera positions and orientations. The camera pose when capturing the images may be determined based on matching the teeth or other features in the 2D projections with corresponding features in the captured images.

In some embodiments, the captured images may be analyzed or processed to identify the teeth in the image. For example, a neural network or other classifier may identify the teeth in the images. A camera pose may be initially estimated based on the identified teeth or other anatomy in the image. Then, the camera pose may be refined based on the key points in the images.

Triangulation may then be used to determine the location of the features in the images and a point cloud may be generated. In some embodiments, the generation of the point cloud may occur during (e.g., simultaneously with) the camera pose determination process.

The point cloud may then undergo bundle adjustment, wherein the 3D coordinates representing the scene geometry and the parameters of the cameras capturing the images (e.g., position, orientation, and intrinsic parameters such as focal length and lens distortion) are refined. Bundle adjustment may include adjusting the 3D points and camera parameters of the camera pose to minimize the re-projection error, which is the difference between the observed image points and the projected points derived from the 3D model and camera parameters. The bundle adjustment may include a least squares optimization.

In some embodiments, the structure from motion model and the point cloud generated using the structure from motion process may be used to generate 2D images from novel viewpoints not found in the original images.

In some embodiments, the 2D image generated using the structure from motion process may include noise or other defects, such as holes or missing information. A machine learning model, such as a generative adversarial network or a diffusion model, may be trained on the patient dentition data, which may include the original video or images captured of the patient and/or 3D scan data or generic dentition data. The machine learning model may then be used to corrects the defects, such as by filling in holes or other missing information.

FIG. 1(b) is a flowchart or flow diagram illustrating a method that may be performed for evaluation of the progress of a treatment plan in an implementation of an embodiment of the disclosed system and methods. The flowchart presents one example use of the techniques or approach disclosed and/or described herein, and others are possible and may be used to develop and/or monitor the progress of a treatment plan for a patient.

As shown in the FIG. 1(b), in one embodiment, a process or method for monitoring the progress of a treatment plan for a patient may include one or more of the following steps, stages, operations, or functions. In some embodiments, at block 120, multiple images (e.g., photos, video) of the patient's dentition, including the teeth and gums may be received. These images may have been captured at a first time T1 (e.g., during a first stage in a dental treatment) using any suitable device (e.g., a mobile device, a portable scanner capable of capturing images). Along with the image data of a static or dynamic subject or scene and, one or more of other data related to the capture, such as camera position, orientation, and other settings may be received in some embodiments. In some embodiments, the images or video may be captured by a patient using their mobile device (e.g., as part of a virtual monitoring program), and the set of images may be associated with data or metadata describing camera settings, camera positioning and orientation, and in some cases, environmental data. The images and associated data or metadata may be provided to a remote platform (in one embodiment) for further processing and evaluation.

In some embodiments, images (e.g., photos, video) captured at different times may be used, such as at a time, T1, and a time, T2, that is after T1. While some anatomical features may move between times T1 and T2, non-moving anatomical features captured in the images at T1 and T2 may be used to align images taken at different times. In some embodiments, features that are not expected to move may be used to align images. For example, in an orthodontic treatment plan, some teeth, such as distal molars, may remain stationary and act as anchor teeth during some stages of treatment while anterior teeth, such as incisors are repositioned. Such anchor teeth or other teeth that have no planned movement during or between T1 and T2 may be used to align images captured at T1 and T2. In some embodiments, the non-moving features and their relative positions within an image may be used to derive the position and orientation of the camera or other imaging device at the time of image capture.

Non-moving features include features that are not planned to move during a treatment plan and may also include teeth that are not treated, such as during restorative procedures. When capturing a dynamic subject in a scene, such as capturing bite, chewing, jaw articulation, changing facial expressions, etc., the subject may be moving, but the non-moving features discussed herein may be used to align the images or videos.

At block 122, the received images (e.g., photos, videos) may be filtered or sampled to create a set of images of sufficient quality (e.g., Input to Trained Model, Evaluate Blur, Combine Multiple Images, or Focus Stacking). The received images may be evaluated for image quality using one or more techniques. For example, captured images may be input into a trained model to evaluate the images for quality. The trained model may output a quality metric for each image, may output an indication of which images are of acceptable quality, or may output images of acceptable quality. In some embodiments, portions of images may be evaluated for quality by the trained model. The trained model may indicate which portion of images are of acceptable or unacceptable quality, may assign a quality metric to each portion, or may otherwise indicate which positions of images are of acceptable and/or unacceptable. Similarly, the blur in images or portions thereof may be evaluated. For example, captured images may be input into a trained model to evaluate the images for blur. The trained model may output a blur or sharpness metric for each image, may output an indication of which images are of acceptable blur or sharpness, or may output images of acceptable blur. In some embodiments, portions of images may be evaluated for blur by the trained model. The trained model may indicate which portion of images are of acceptable or unacceptable blur, may assign a blur metric to each portion, or may otherwise indicate which positions of images are of acceptable and/or unacceptable.

In some embodiments, images or portions thereof, such as portions with acceptable quality and/or blur may be combined such as through focus stacking, or to expand the view of the image to include more of the patient's anatomy to produce an image or a set of images with suitable characteristics for use. The images may be processed to enhance features, contrast, remove glare, or alter another aspect of an image based on the associated data or metadata.

At block 124, a reconstructed representation of the patient's dentition may be created. As described elsewhere herein, a reconstructed representation of a patient's mouth may be generated using one of several techniques or approaches (e.g., including differential volumetric rendering approaches such as NeRF and Gaussian Splatting).

At block 126, an image (e.g., photos, video) of a patient's teeth may be taken at time T2, where T2 is after T1, and may be received with or without camera position, orientation, and settings indicated. This may include the patient capturing an image of their mouth with a set of associated cameras and/or environmental data or metadata. In some embodiments, the camera position and orientation may be determined based on the image data, rather than being provided with the image.

The previously constructed representation of the patient's mouth may then be used to generate a suitable image (e.g., photos, video) for comparison to the more recently provided image. For example, at block 128, a reconstructed representation of the patient's teeth may be accessed. As another example, a dynamic reconstructed video representation of a patient opening and closing their jaws may be accessed. The previously generated representation may be stored locally on a mobile device and/or on the remote platform.

At block 130, an image (e.g., photos, video) of the patient's teeth may be generated using the reconstructed representation based on one or more of camera position, orientation, and settings for the image captured at time T2. The reconstructed representation is used to generate a novel image (e.g., photos, video) with camera position, camera settings, and/or environmental conditions similar to that associated with the image taken at time T2 using one or more of the methods discussed herein.

At block 132, the image (e.g., photos, video) of patient's teeth generated using the reconstructed representation is compared to the image taken at Time T2. The image generated using the reconstructed representation is compared to the image taken at time T2 using a human or trained model to identify differences or any areas of concern in the image. As suggested, this may be performed manually and/or with the assistance of a trained model that identifies differences in the two images.

Other types of view may be generated and comparisons made. For example, in some embodiments, an original image (e.g., photos, video) captured at time T1 and a synthesized image (e.g., photos, video) generated based on images (e.g., photos, video) captured at time T2, where the T2 image is synthesized to correspond to the same position and orientation as the original T1 image may be used for identifying changes in the patient's anatomy for dental diagnosis, tracking, or treatment. In some embodiments, a synthesized image generated based on images captured at time T1 and an original image captured at time T2, where the T1 image is synthesized to correspond to the same position and orientation as the original T2 image may be used for identifying changes in the patient's anatomy for dental diagnosis, tracking, or treatment.

In some embodiments, a first synthesized image (e.g., photos, video) may be generated based on images captured at time T1 and a second synthesized image may be generated based on images captured at time T2. The camera position, orientation, field of view, etc., of both the first and second image may be the same. The first and second synthesized images may be used for identifying changes in the patient's anatomy for dental diagnosis, tracking, or treatment.

In some embodiments, the synthesized image may not be generated based on a virtual camera view having a focal length, field of view, focus distance, or other camera properties. For example, in some embodiments, a synthesized or novel view may be generated as a panoramic images, that unrolls the dental arch and provides visibility of all teeth in a single image. Such panoramic or unwrapped images can be helpful for comparisons, evaluations, diagnosis, and track.

At block 134, based on the comparison, the treatment plan may be modified or a further treatment may be determined to be necessary or optimal. The modification or further treatment may then be recommended to a dental professional and/or the patient (e.g., via a notification on an interface of a software application). For example, if the teeth or jaw are not moving as expected, a course correction may be recommended (e.g., with a new set of aligners). If gingival defects or other dental or oral health issues are identified as likely, an intervention may be recommended. In some embodiments, jaw articulation, chewing motion, or bite function may not be as expected after restorative work has been preformed. Based on the comparison, the existing plan may be modified or maintained at the discretion of the dentist or orthodontist.

In some embodiments, a comparison may be made between the position of the teeth in the captured images and/or the reconstructed representation and an expected position of the teeth, such as in a treatment plan. If the positions do not sufficiently match, an orthodontic treatment may be off track. A new orthodontic treatment plan may be generated to move the teeth from the current position towards a desired final position. In addition, new orthodontic appliances, such as aligners may be fabricated. The fabricated aligners may be worn by the patient.

As described, during the image or video capture process performed using a patient's mobile device, an application installed on the device may control aspects of the operation of the device's camera. This may be done to improve the quality or utility of the captured images and compensate for limitations of the device's camera. For example, optical limitations such as a short focus distance may create a shallow depth of field in which only certain portions of an image are in focus at a given time. Also, the camera's flash is typically positioned near the subject teeth which may mean that only certain portions of an image or video are properly exposed. Finally, to fully capture the arch of the mouth, the patient may take multiple images (e.g., photos, video) which increases the time and care required to obtain the images.

To compensate or correct for such limitations, in some embodiments, multiple images may be combined into a single composite image. In one such embodiment, an application on the mobile device may control the camera to capture the desired set of images. In an example use case, a mobile device captures multiple photos. The set or sets of images are either processed on the mobile device or are uploaded to a server for processing. Once processed, one or more composite images may be produced which can be analyzed by a dental practitioner and optionally assessed through an AI/ML based mechanism, or used as input to generate one or more of the reconstruction models discussed herein, such as differential volumetric rendering models including neural radiance fields and Gaussian Splatting, structure from motion, diffusion, etc.

Approaches for the generation of a composite image from multiple captured images are described with reference to FIG. 1(c), and may include focus stacking, high dynamic range (HDR) images with tone mapping, and/or generating panoramic images of the dental arch. While reference is made to photos or images, the disclosure may be applied to videos, such as for example, the frames of a video. For example, videos of a moving subject may be captured simultaneously from different viewpoints to allow for stitching into a video panorama, at sufficiently high frame rates with differing exposure or focus values to allow for focus stacked or HDR video generation.

Focus stacking refers to a process used to compensate or correct for the depth of field (DoF) limitations of many mobile device cameras. Depth of Field (DoF) is the range of distances from a camera that will be in acceptable focus when the camera is focused at a particular distance. An approximation to a camera's true depth of field can be represented as

$$DOF \approx \frac{2 {u}^{2} Nc}{{f}^{2}} ,$$

where u is the distance to the subject, N is the f-number or “f-stop” representing the focal ration of the lens when taking the image, f is the focal length, and c is the circle of confusion. Due to the inherent properties of a mobile device camera, the circle of confusion (which is proportional to the size of the camera's sensor) is typically very small. For reasonable assumptions of the relevant parameters, when focusing on an object that is 4 inches away, a rough distance at which oral images may be captured, the depth of field can be as small as 0.5 inches, meaning that objects between 3.75 inches and 4.25 inches away from the camera lens may be in “reasonable” focus. When the camera is closer to the dentition, the depth of field may be even smaller, such as less than ¼ or ⅛ of an inch.

To address this limitation, in one embodiment, the camera of a device used to capture dental images (e.g., a patient's mobile device) may be configured to collect multiple photos at varying focus distances. The photos can then be aligned using a point matching approach to find a suitable transform that relates the photos (affine, homography, or linear motion, as examples). Once the images are aligned, they can be combined through a focus-stacking algorithm (such as the complex wavelet-based approach). Other approaches are also possible as part of this approach, such as identifying which image has the maximum amount of variance in the observed edges. By focus stacking multiple images taken form the same or similar viewpoint, a single composite image may be generated form that viewpoint with a depth of field greater than any single image used to generate the composite. This process may be repeated for images taken from multiple viewpoints. The composite images from the multiple viewpoints may then be used as input into one of the reconstruction methods discussed herein.

In some embodiments, high dynamic range (HDR) images with tone mapping may be used to address, e.g., some of the camera limitations discussed above. In these embodiments, a camera device may take multiple images at different photographic exposures when capturing a view, and a composite image may be created from those images. casein some embodiments, in addition to or instead of collecting images at varying focal depths, the device is instead operated (typically by an installed application or instruction set) to collect images at different exposure sensitivities. Because the camera is typically positioned closer to some of the teeth in an image as compared to other teeth in the image, the camera's auto-exposure algorithm may be set so the camera functions to properly expose only the nearest teeth (e.g., teeth within a threshold radius or range), with the remainder of the image frequently being under-exposed. By varying the exposure sensitivity and/or exposure parameters, such as shutter speed and aperture, the process can capture multiple images, each with a different portion of the dental cavity and with a desired exposure.

After aligning the images, a single composite image can be made with most (if not all) regions of the dental cavity having proper (or at least suitable) exposure. This increases the suitability of the image(s) for dental assessment by either an AI/ML algorithm or direct visual assessment by a trained clinician. This approach optimizes the tone mapping such that various portions of the image are properly exposed. This process may be repeated for images taken from multiple viewpoints. The composite images from the multiple viewpoints may then be used as input into one of the reconstruction methods discussed herein.

In some embodiments, the methods and systems disclosed herein may be configured to capture a series of photos taken along the dental arch of a patient to capture images of teeth along the arch. Once the photos have been captured, a single panoramic image can be constructed which provides the dental practitioner with a single image (e.g., a single continuous image) displaying, for example, the entire dental arch (e.g., the buccal sides of all the patient's teeth, the lingual sides of all the patient's teeth, and/or the occlusal sides of all the patient's teeth.

In some embodiments, a composite image may be a focus stacked and high dynamic range tone mapped image, wherein multiple images of varying focus and exposure from the same or similar viewpoint may be combined through the processes described herein. This process may be repeated for images taken from multiple viewpoints. The composite images from the multiple viewpoints may then be used as input into one of the reconstruction methods discussed herein.

In some embodiments, posterior teeth that may not be clearly visible are captured at a quality sufficient for generating reconstruction of some or all posterior teeth. In some embodiments, the reconstruction may be based on the anterior teeth. To generate a full set of teeth for the reconstruction, a 3D model of the patient's teeth, such as from a 3D scan of the patient's teeth, may be aligned with the anterior teeth of the reconstruction. The position and orientation of the posterior teeth in the aligned 3D model may be used for the posterior teeth in the reconstruction.

FIG. 1(c) is a flowchart or flow diagram illustrating a process, method, operation, or function that may be performed for Image Capture or Image Quality Assessment of images (e.g., still or video) in an implementation of an embodiment of the disclosed system and methods. The flowchart presents one example use of the techniques or approach disclosed and/or described herein, and others are possible and may be used to develop a dataset for training a model or evaluating the state of a treatment plan by creating a reconstructed representation of a patient's mouth and teeth. While reference is made to images, the disclosure may be used with still or video images of static or dynamic subjects.

As shown in the figure, image capture may include one or more of the following steps, stages, operations, or functions. At block 140, an application to control image capture in a mobile device, may be installed. The application may be downloaded from a remote platform and installed by a patient in their mobile device. In one embodiment, the application may generate a user interface and/or audio to assist a patient in capturing the desired images.

When instructed, the patient may use the camera to capture one or more images, such as a still image or video. In one embodiment, the installed application may control the mobile device camera as follows:
Use the application to control the camera to capture multiple images of the patient's dentition at varying focal distances (depth of field) at one or more viewpoints along the dental arch, at block 142.
Use the application to control the camera to capture multiple images of the patient's dentition varying exposures at one or more viewpoints along the dental arch, at block 142.
Use the application to control the camera to capture multiple images of the patient's dentition along the dental arch (panoramic) at block 146.

At block 148 the captured image or images are provided to the remote platform for processing and/or evaluation. The images may be processed to generate composite images, such as focus stated and/or high dynamic range and/or panoramic images. The captured and/or composite images may be used to create a reconstructed representation of patient's dentition. Given sufficient images and images of sufficient quality, the disclosed and/or described reconstructed representation of the patient's mouth and teeth is created.

As disclosed and/or described herein, embodiments may perform an evaluation of image quality to determine if an image provided by a patient is suitable for use as part of a dataset for training a model and/or for evaluation of the state of a treatment plan. As shown in the figure, image quality assessment may involve one or more of the following steps, stages, operations, or functions.

At block 150, a captured image quality evaluation processing is initiated. At block 152, images of the patient's dentition are captured as still, video, or a combination of still and video images.

One or more quality assessment processes or operations may be performed on each of the captured photos or composite photos or videos generated from the captured photos or video. For example, at block 154 the images may be input into a trained quality assessment model. The model output may be a quality metric or evaluation.

In another example, the images may be progressively and iteratively blurred, such as at block 156. With each blur iteration, a comparison may be made between the blurriness of the image in the current iteration with the blurriness of the image in the previous or original iteration. Greater reduction in relative blurriness between iterations, such as the first iteration and the original image indicate a sharper image. A blur metric may be determined based on the relative blurriness of an image after one or more iterations, as discussed herein.

By use of the successive blurring technique referred to herein. In this process, an original image is subjected to successive applications of a blurring process or filter (as suggested by FIG. 1(c)). Each iteration of the blurred image is evaluated to determine a characteristic metric. It has been found that the value of the metric will change less between the successive iterations if the original image was relatively more blurred, and therefore of lower image quality.

Based on this assessment, an original, sharper image may be used with greater confidence as part of the reconstruction representation and/or as part of a set of training data.

An image of sufficient quality may also be used as a starting point to generate multiple related images through input of a set of changing parameters or instructions to an image generator (such as one based on a generative technique and/or the reconstruction approach disclosed and/or described herein).

A non-limiting example of suitable blur filters and blur metric may include processing the image with a Gaussian blur filter, such as:

$$G ( I , x , y ) = {\sum }_{{x}^{′} , {y}^{′}} \frac{I ( {x}^{′} , {y}^{′} )}{\sqrt{2 π {σ}^{2}}} {e}^{- \frac{{( x - {x}^{′} )}^{2} + {( y - {y}^{′} )}^{2}}{2 {σ}^{2}}}$$

The blurred filter may then have a Laplacian filter applied, such as:

$$L ( I , x , y ) = \frac{{\partial }^{2} I}{\partial {x}^{2}} + \frac{{\partial }^{2} I}{\partial {y}^{2}}$$

This results in a Laplacian of Gaussian filter, such as:

LOG I)=L(G(I,x,y),x,y),

In these equations, I denotes pixel intensity and x and y are the columns and rows of each pixel in the image. The Laplacian of Gaussian is a representation of the blurriness of a given pixel for a particular blur iteration. Examples of a blur metric for a pixel may include the terminal Laplacian of Gaussian, such as the value of the Laplacian of Gaussian after a certain number of iterations, such as 10 or 20 or 100 iterations, the slope of the curve of the Laplacian of Gaussian at the very first or first few iterations, or the area under a curve of the Laplacian of Gaussian after a certain number of iterations, such as 10 or 20 or 100 iterations.

The blur metric may be evaluated for an individual pixel or pixels to determine the relative blurriness of a single pixel or pixels, or the relative blurriness of a single pixel as compared to another single pixel. In some embodiments, the blur metric may compare one region of an image (a group of pixels, such as a group of adjacent pixels) with another region of the image, such as by averaging the blurriness metric for each of the pixels in each respective region. In some embodiments, the relative blurriness of multiple images may be compared based on an average of the blurriness metric for each respective image.

FIG. 1(e) depicts an illustration of a process 170 for iteratively blurring an image to determine its blurriness, as discussed herein, and an associated feature curve 180, which is a curve of the blurriness measure as a function of iterations, for an example image. At block 172 an initial image A is provided. In the column of blocks 174 images are blurred, in the column of blocks 176 a blur metric for each feature in the image is determined. In some embodiments, a feature in the image may be an anatomical feature, such as a tooth, an interproximal area between teeth, a portion of the image, a portion of an anatomical feature, or a single pixel or group of pixels in the image. In the column of blocks 178, the blur metric for each feature in the image is aggregated. The blur metric, such as the aggregate blur metric is shown in the plot 180 in which the horizontal axis represents the iterations and the vertical axis is the blurriness metric, such as the aggregate blur metric from column 178. Each row in the process 170 represents an iteration of the blur process. Each time the image is further blurred, the blur metric and aggregate blur metric is determined for that blurred image and the resulting blurriness measure is shown plotted on the curve 180.

In some embodiments, the blur metric and the above iteration method may be used to train an image quality machine learning model, such as the model used at block 154 in FIG. 1(c). For example, a machine learning model may be trained using a set of images and corresponding blurriness metric or curve, as shown and described herein, such as with respect to FIG. 1(e). In some embodiments, a human assessment as to the blurriness of the image may also be included, such as a blurriness score on a scale of 1 to 10. The trained machine learning model may then be provided with, for example, a new image. The output of the model may be a blurriness metric of the new image or a blurriness score, such as on a scale of 1 to 10 or a Boolean, such as an indication whether or not the new image is sufficiently sharp for use or whether or not the new image is sufficiently blurry as to not be used.

Additionally, or instead, supervised (e.g., logistic regression, trees, random forest, with or without PCA) or unsupervised classification (e.g., clustering) models can be used to identify blurry as compared to sharp images.

As shown at block 155, another method of image quality assessment may be used. For example, an agreement-based image quality assessment may be used. A set of previously obtained images may have one or more types of features within the image labeled. Such a labeled dataset may be available from an operator of a service to provide the functionality disclosed and/or described herein, who may have access to images captured by multiple patients (which may be used after suitable anonymizing). Such images and associated labels may be generated by one or more of a programmatic labeling model, a human labeler, or other validation source of suitable for the purpose. In some embodiments, the labels may also include a confidence value that indicates the confidence the labeler had with their determination that the labeled feature is present in the image at the labeled location.

In general, identifying photos or regions of photos with image quality issues is a subjective, labor-intensive, and often task-dependent process (as an image considered blurry for one task may be suitable for another). Supervised machine learning uses a large amount of labeled data to train models and in many cases, there are multiple sources of truth for images. As mentioned, in one embodiment, indications of agreement/disagreement between these different sources may be used to train a model to identify labeling errors and, in some cases, estimate/predict image quality.

Image quality assessment based on agreement between multiple validation sources may provide one or more of the benefits. In some embodiments, image quality assessment allows for an image quality to be estimates as a mask across an image. As described further herein, images may be portioned or segmented into regions near features that have been labeled by the multiple validation sources. For example, bounding boxes or regions may be formed around each of one or more attachments (or some other feature that has been labeled by the multiple validation sources), and disagreement on the presence of each attachment may be indicative of quality only on the bounding boxes or regions within which the attachment rests. A mask may then be applied to the respective image indicating which bounding regions are likely above a threshold quality level (e.g., having sufficient agreement among multiple validation sources) and which bounding regions are likely below a threshold quality level (e.g., having insufficient agreement among multiple validation sources). In embodiments, an overall image quality for an image may be estimated based on the relative amount (e.g., ratio, percent) of unmasked regions in the image (e.g., the regions above the threshold quality level) as compared to masked regions in the image (e.g., the regions above the threshold quality level). In other embodiments, the unmasked regions may be relied upon (e.g., for training a machine learning model, for determining whether an image is acceptable, etc.), and the masked regions may be discarded or distrusted.

Although attachments are discussed herein, they are just one type of appliance structure. Image quality assessment based on agreement between multiple validation sources my include labeling of other appliance structures, such as buttons, auxiliaries, or other structures located on the patient's teeth or other tissue. In some embodiments, appliance structures may be located on an appliance. For example, the structures may be projections or grooves placed on an appliance for the explicit purpose of identifying good and bad image or assessing the quality of an image or portion thereof.

In some embodiments, a mask can be created that indicates the quality across an image. For example, each location in the mask, such as a pixel of the mask, may be assigned a value based on the relative quality of the image at that location.

In some embodiments, labeled images may be used to train a machine learning or artificial intelligence algorithm. For example, images may be labeled by multiple people for the presence of attachments, where agreement between labelers indicates a quality image or portion of an image and disagreement between labelers indicates a lack of quality or insufficient quality of an image or portion of an image. A model trained using these labeled images may then be used to determine the quality of images, even without labels.

In some embodiments, image quality assessment allows for determining quality by leveraging existing information (e.g., labelling of attachments, which may have been part of a treatment analysis/monitoring of individual patients) In some embodiments, the information may be leveraged without further time or effort to be spent specifically on quality review.

In some embodiments, image quality assessment allows image quality estimates to be customized for particular applications. For example, in some embodiments, assessments as to agreement of a task may be used for a particular application. An example provided herein is the task of labeling attachments in an image with an application of image quality assessment. Here, the task of labeling attachments in images aids in determining the location of attachments in the image. However, when the same image or multiple images of the same dentition are labeled with attachment locations, agreement or disagreement between may be an indication of quality of the image or images, where agreement indicates a higher relative quality than disagreement.

Currently, there is no way practical to perform image quality estimation across diverse image types. Training such a model may be possible, but would be expected to require slow, expensive, and error-prone labeling efforts. Labeling image quality across an image, such as a mask giving image quality for each pixel value or group of pixels, would be useful, but may not be practical to achieve. However, a way to address these shortcomings is disclosed and/or described in the following.

Image quality can have a significant impact on labeling accuracy and inter-labeler agreement. If labels are whole image based (e.g., a classification of an image), then images with a disagreement between labels generated by different processes are more likely to result from images with lower quality. If the labels are region based (e.g., they are processed to indicate bounding boxes or segmentation labels), then the regions of the images containing disagreements are more likely to result from lower image quality in those regions.

In some cases, labeling agreement or disagreement can be assessed using one or more of the following types of comparisons:
Comparing labels of the same image across different labelers. Such inter labeler agreement may be used as discussed herein to determine the quality of an image or portion thereof. For example, labels may be added to images or portions therefore to identify features or attributes in or of the image by multiple labelers. Agreement or disagreement between the labels of in image or portion thereof may be an indication of quality of the image or portions thereof, where agreement indicates a higher relative quality than disagreement.
Comparing labels to a trusted validation source, such as a known patient treatment plan. In some embodiments a rusted validation source may be used in a comparison with labelled images. For example, a treatment plan may indicate the locations of attachments or other features in or on a patient's dentition. Photos of the patient's dentition may be labelled by labelers with the locations or attachments or other features in the image. If the labelled images match the treatment plan data, then the images may be of an acceptably high quality. If the labelled images do not match the treatment plan then the images may be of an unacceptable or low quality.
Comparing labels, such as from a human labeler, to machine learning model predictions, with predictions obtained from a model trained with a different dataset or using a cross-validation technique. For example, in cross validation of a machine learning model for predicting tooth numbering. In this case, label-prediction mismatches between a model's prediction and a label may be due to model prediction errors and not labeling mistakes. If this happens, the model may learn to predict images that are difficult to segment (because the labeling each tooth or pixel in an image with a tooth number may be part of the segmentation process) as having lower quality. Such mismatch between the label and the prediction may occur, for example, in dentitions with severe anterior occlusion.

In each of these cases, images or regions of images with disagreements between different labeling assessments are labeled as such. A machine learning model is then trained to learn these labeling “mistakes”. Note that in implementing this approach, care should be taken to ensure that labeling mistakes are not confused with some other aspect of the image.

As an example, mixed dentition may have more labeling mistakes for certain tasks and have lower image quality, particularly if the images are taken by children. Care should also be taken so that the validation source does not have a bias that could influence the predicted image quality assessment. An example in this case might be using cross validation of a machine learning model for predicting tooth numbering as the source of truth. In this case, label-prediction mismatches may often be due to model prediction errors and not labeling mistakes. If this happens, the model may learn to predict images that are difficult to segment as having lower quality (e.g., severe anterior occlusion).

An example of how this approach could be used in practice involves assessing agreement among validation sources (e.g., human labelers, a programmatic labeling model, a known treatment plan of a patient in question) in their determination of the presence of a dental or orthodontic attachment, another auxiliary (e.g., a button, a power arm), or other feature. Attachments are sometimes small, and often tooth colored, rendering them nearly invisible if image quality is not sufficiently high. Even when photos are provided as a photo set with multiple angles, and 3D models with attachments are available to labelers, many photo sets may have one or more teeth whose attachments are not visible, and may thus not be determined as having such attachments, even though they may be actually present on the patient's teeth. In some embodiments, the determinations can be evaluated against a treatment plan of the patient to determine if the treatment plan “agrees” with the labeler. For example, a labeler may not label a particular tooth of a patient as having an attachment, but the treatment plan of the patient may indicate that an attachment should be present on the tooth. Although it is possible that the attachment may have fallen off, the “disagreement” between the labeler and the treatment plan may indicate that the image is of low quality (or at least that the portion of the image near the attachment region is of low quality)—e.g., the inference being that the labeler did not see the attachment due to low image quality. As another example, determinations among multiple labelers (e.g., human labelers and/or programmatic labelers) may similarly be compared for disagreements. And likewise, disagreements may be treated as evidencing low image quality (of the image as a whole or of the portion of the image near the attachment or other feature in question).

Likewise, teeth in regions of the image with relatively high quality may have attachments that match the treatment plan, and may thus be said to “agree” more with the treatment plan. Similarly, agreement among multiple validation sources (e.g., multiple human labelers and/or programmatic labelers) can be an indication of relatively high quality. If regions of an image having high and low quality can be identified in the image, such as by comparison of how well teeth attachment labels match a treatment plan, then a mask may be created showing the regions of relatively higher and lower quality across the image. By repeating this across all attachment labels, a set of image/image quality mask pairs may be constructed that can be used for training a semantic segmentation model. The semantic segmentation model is then trained to predict where regions of lower quality might be in an image.

In some embodiments, the possibility of attachments falling off during treatment may be accounted for in the training and use of a quality assessment that employs these methods so as not to confuse the visual presence of a missing attachment with an improperly tagged image. One way this problem can be mitigated is by using the observation that teeth more distal in an image are often blurrier than teeth closer to the camera (which is often the focus point). Due to this situation, when an attachment label is missing on a tooth, that tooth and any tooth more distal to that tooth in the image may be labeled as being in a lower quality region of the image.

Likewise, images with teeth that have all their attachments labeled (as determined based on agreement with a treatment plan and/or other validation sources such as labelers) can be considered to be in regions of the image having relatively higher quality. In a similar way, a tooth more anterior to these teeth would also be considered to be in a higher quality location of the image. Some teeth may not have attachments and might be located between a tooth with missing attachment labels and another tooth with attachment labels present. In this case, the image quality may not be known for this tooth with sufficient accuracy.

In one embodiment, pixels for teeth in regions of relatively higher quality could be labeled with a ‘1’ and those in regions of relatively lower quality with a ‘-1’. Regions outside of teeth, as well as those teeth in regions of unknown/ambiguous quality would be labeled as ‘0’. A neural network may receive an image as input, and produce a mask with the same dimensions as the input. Parameters of the network could learn to output higher (or lower) values for regions of the image with higher (or lower) relative quality.

A similar approach can be used where an image has been labeled by multiple labelers. In this case, images (or regions of images) where the labelers disagree are more likely to be from images/regions with lower image quality. However, having multiple labelers label a single image is not cost effective. An alternative approach uses labeled data to train a cross-validated machine learning (ML) model. The model predictions on “hold out” data can be compared to their labels. In this case, regions with higher levels of inaccurate predictions may indicate poor quality images or poorer quality regions of images.

In one embodiment, multiple of the disclosed and/or described techniques may be used together. For example, images at different camera orientations and positions may be used to generate a model of a patient's mouth and teeth using a suitable reconstruction technique. This may include use of multiple focus distance for capture of multiple images. Next, an evaluation of a blurriness metric and/or other quality metric may be used to determine whether focus stacking is necessary for one or more images and apply focus stacking to the images where indicated. Next, compare one or more qualities of a set of focus stacked images to non-focus stacked images and select the most useful for a specific training or inference task.

At block 158 as set of images with acceptable quality may be used to train or generate a model or models discussed herein, such as for use in generating a reconstructed representation.

FIG. 1(g) depicts a flowchart or flow diagram illustrating a process, method, operation, or function that may be performed for generating and manipulating a morphable 3D model using dynamic differential volumetric rendering processes, such as the dynamic NeRF and dynamic Gaussian Splatting processes discussed herein. Dynamic differential volumetric rendering is a rendering technique that extends differential volumetric rendering by incorporating time-based variations, allowing for the rendering of dynamic, evolving scenes. Dynamic differential volumetric rendering captures changes to a scene that occurs over time, enabling novel view representations of dynamic scenes. A morphable 3D model allows may be a parametric representation of a 3D object or objects, such as the teeth and jaws of a patient that can be modified by adjusting predefined parameters, such as the parameters discussed below.

At block 710 video of a patient moving their jaws and face through various expressions, poses, and/or positions are captured. In some embodiments, the positions may include positions 750 including a neutral bite, lateral right bite, lateral left bite, retraction bite, protrusion bite, and open bite. Expressions may include facial expressions such as neutral expressions, open mouth smiling, closed mouth smiling, etc. Poses may include the pose of the face or head.

At block 720 a dynamic differential volumetric rendering model is generated. The dynamic differential volumetric rendering model may be generated using any of the methods described herein. The model may be generated with additional parameters as compared to the models described herein. For example, each pixel, Gaussian, etc., may be assigned additional parameters for each time in a video. The additional parameters may include parameters related to expression, poses, and/or positions.

At block 730 desired views, poses, expressions, positions, etc. may be received or evaluated. For example, in some embodiments, standard views, poses, expressions, positions, etc., may have been previously generated using a generic model; a 3D model of the patient's teeth, jaws, and other anatomy; or another model may be received for use in generating a novel view. In some embodiments, the desired views, poses, positions, expressions, etc. may come from a video or still images of another person or patient in standard views, poses, expressions, etc. The views, poses, positions, and expression parameters may be generated based on the views, poses, positions, expressions, etc., in the model, images, etc. In some embodiments, the view views, poses, expressions, positions, etc., either standard or generated from a model, video, etc, may be evaluated and a set or sets of parameters selected for use in generated novel views.

At block 740 the views, poses, positions, and expression parameters may be used as input into the dynamic differential volumetric rendering model of block 720 to generate novel views 760 based on the patient video captured at block 710 using the desired views, poses, positions, expressions of block 730. Novel view 760 may be an image or video showing a patient moving the jaw in a standard movement from a standard viewpoint. For example, the camera used to capture the video at block 710 may not have been in a clinically relevant location and/or the patient may not have moved or posed in a clinically relevant manner. The dynamic differential volumetric rendering model of block 720 and the views, poses, positions, and expression of block 730 may be used to generate the clinically relevant data.

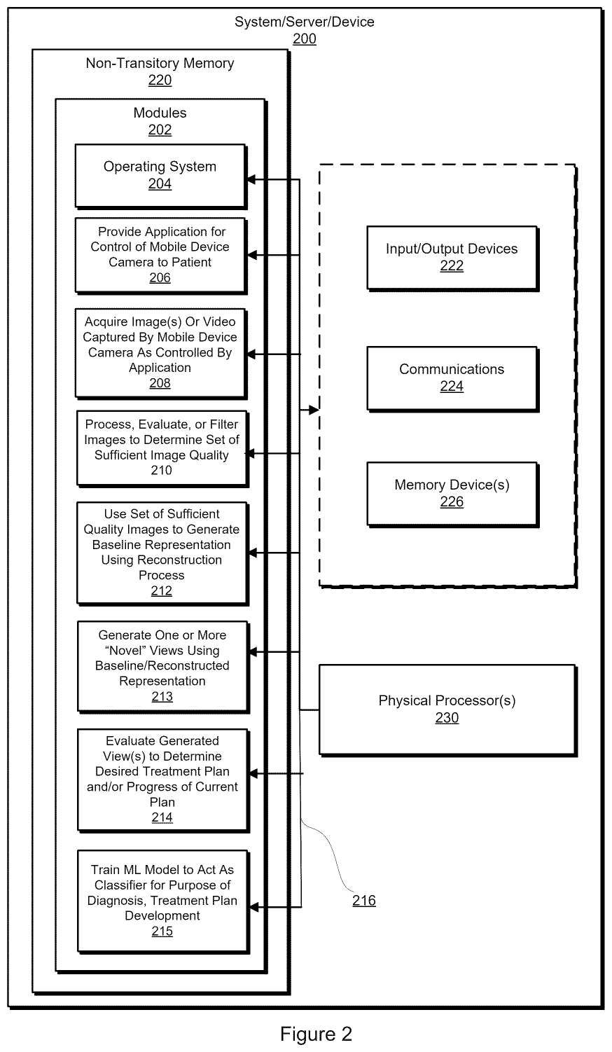

FIG. 2 is a diagram illustrating elements or components that may be present in a computer device or system configured to implement a method, process, function, or operation in accordance with an embodiment of the system and methods disclosed herein. As noted, in some embodiments, the system and methods may be implemented in the form of an apparatus that includes a processing element and set of executable instructions. The executable instructions may be part of a software application and arranged into a software architecture.

In general, an embodiment may be implemented using a set of software instructions that are designed to be executed by a suitably programmed processing element (such as a GPU, CPU, TPU, QPU, microprocessor, processor, co-processor, or controller, as non-limiting examples). In a complex application or system such instructions are typically arranged into “modules” with each such module typically performing a specific task, process, function, or operation. The entire set of modules may be controlled or coordinated in their operation by an operating system (OS) or other form of organizational platform.

Each application module or sub-module may correspond to a particular function, method, process, or operation that is implemented by the module or sub-module. Such function, method, process, or operation may include those used to implement one or more aspects of the disclosed and/or described systems, apparatuses, and methods.

The application modules and/or sub-modules may include a suitable computer-executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language.

The modules may contain one or more sets of instructions for performing a method, operation, or function described with reference to the Figures, and the disclosure and descriptions of the functions and operations provided in the specification. These modules may include those illustrated but may also include a greater number or fewer number than those illustrated. As mentioned, each module may contain a set of computer-executable instructions. The set of instructions may be executed by a programmed processor contained in a server, client device, network element, system, platform, or other component.

A module or sub-module may contain instructions that are executed by a processor contained in more than one of a server, client device, network element, system, platform, or other component. Thus, in some embodiments, a plurality of electronic processors, with each being part of a separate device, server, or system may be responsible for executing all or a portion of the software instructions contained in an illustrated module or sub-module. Thus, although FIG. 2 illustrates a set of modules which taken together perform multiple functions or operations, these functions or operations may be performed by different devices or system elements, with certain of the modules/sub-modules (or instructions contained in those modules/sub-modules) being associated with those devices or system elements.

As shown in FIG. 2, system 200 may represent a server or other form of computing or data processing system, platform, or device. Modules (or sub-modules) 202 each contain a set of executable instructions, where when the set of instructions is executed by a suitable electronic processor or processors (such as that indicated in the figure by “Physical Processor(s) 230”), system (or server, platform, or device) 200 operates to perform a specific process, operation, function, or method.

Modules 202 are stored in a (non-transitory) memory 220, which typically includes an Operating System module 204 that contains instructions used (among other functions) to access and control the execution of the instructions contained in other modules. The modules 202 stored in memory 220 are accessed for purposes of transferring data and executing instructions by use of a “bus” or communications line 216, which also serves to permit processor(s) 230 to communicate with the modules for purposes of accessing and executing a set of instructions. Bus or communications line 216 also permits processor(s) 230 to interact with other elements of system 200, such as input or output devices 222, communications elements 224 for exchanging data and information with devices external to system 200, and additional memory devices 226.

For example, Modules 202 may contain computer-executable instructions which when executed by a programmed processor cause the processor or a device in which it is implemented to perform the following processes, methods, functions, or operations.

Module 206 may provide application for control of a mobile device camera to the patient. In one embodiment, the application may be obtained by the patient downloading the application from a remote platform or website.

Module 208 may acquire image(s) or video captured by mobile device camera as controlled by an installed application. In one embodiment, the desired timing (such as time of acquisition, or time interval between captured images), location, environmental conditions (lighting, temperature), camera settings (focal length, exposure, illumination, aperture, focus distance), and/or other characteristics of captured images or video may be controlled and/or stored by the application.

In one embodiment the mobile device's position, orientation, or other characteristic(s) relevant to an image or video may be determined and provided to a dentist or orthodontist along with the images or video (such as in the form of meta-data and/or mobile device sensor data). The video and/or images are provided to the patient's dentist or orthodontist, either directly or via their accessing a remote platform (such as a multi-tenant or SaaS platform). If the patient captures a video, that video may later be processed to generate one or more images. This could include sampling or filtering, as non-limiting examples.

Module 210 may process, evaluate, or otherwise filter images to obtain a set of sufficient quality images for use in a reconstruction process. As is disclosed and/or described further herein, this stage of processing may involve one or more of the following for purposes of assessing (and in some cases, correcting) image quality. Detection of poor or insufficient image quality (such as by determining a degree of blur in an image, or whether an initial image may not be correctable). Performing a comparison to previously obtained images to assist in identifying changes in tooth positions or alignment (or misalignment) between images, and/or assist in evaluating the quality of an image provided by a patient. Use of a trained machine learning model to assign a value for image quality or relative image quality.

Module 212 may use the filtered or otherwise evaluated images as inputs to one or more reconstruction processes to generate a baseline representation for synthesis of a novel view or views. Examples of image processing techniques that may be used to generate the reconstructed representation (or fusion) include, but are not limited to: Neural Radiance Fields, Gaussian Splatting, image stitching; structure from motion, and fine-tuned personalized diffusion model.

Module 213 may generate one or more novel views from the reconstructed representation or baseline. The reconstructed representation may be used to generate one or more images that represent the patient's teeth as viewed from a desired perspective, location, environmental condition, or other aspect of interest to the dentist or orthodontist;

Module 214 may evaluate the generated view or views to determine the current state of a patient's teeth and progress of a treatment plan. This process step may be used to monitor the progress of a treatment plan. This step or stage is typically performed manually by a dentist or orthodontist, but may be performed in whole or in part by a trained model.

Module 215 may train a machine learning model to act as a classifier using multiple generated images. This is an optional stage but involves use of multiple generated images to train a model as a classifier for use in diagnosis, development of a treatment plan, or when trained, determination of the state or change in state of a patient's teeth. In one embodiment, the images used as part of a set of training data may be obtained from one or more of the following sources: the reconstructed representation disclosed and/or described herein, when operated under multiple conditions or input parameters, a set of existing images and annotations that have been evaluated to determine their labeling accuracy, and/or a set of images generated by a successive blurring process—in this approach, it is beneficial to determine the change in degree of blurring between images after successive application of a blur filter-if the change indicates that the original image was sufficiently clear, then the original and one or more of the blurred images may be used as part of a set of training data for a machine learning model. Module 215 may also operate the trained model to aid in dental visualization, diagnostics, monitoring, and other dental functions. For example, as discussed herein, the model may be used to create visual representations of a patient's dental anatomy, such as the current state of the patient's teeth from one or more views. In some embodiments, the images may be used in aiding diagnostic applications. Dental diagnostics includes identifying and aiding in treating various oral health issues including diagnosing health issues with teeth, gums, and other structures in the mouth. Dental monitoring may include tracking the progress and status of a patient's dental health and orthodontic treatment over time. For example, the trained model may be used to generate multiple visualizations of the patient's dentition from the same perspective (including field of view and focal length) over time, such as based on multiple imaging sessions spaced out over a period of time. A dental professional may monitor the changes in the patient's gingiva from that same perspective over time and based on, for example, changes in gingival recession, may make a diagnosis. As mentioned, in some embodiments, the systems and methods disclosed and/or described herein may provide services through a Software-as-a-Service (SaaS) or multi-tenant platform. The platform provides access to multiple entities, each with a separate account and associated data storage. Each account may correspond to a dentist or orthodontist, a patient, an entity, a set or category of entities, a set or category of patients, an insurance company, or an organization, for example. Each account may access one or more services, a set of which are instantiated in their account, and which implement one or more of the methods or functions disclosed and/or described herein.

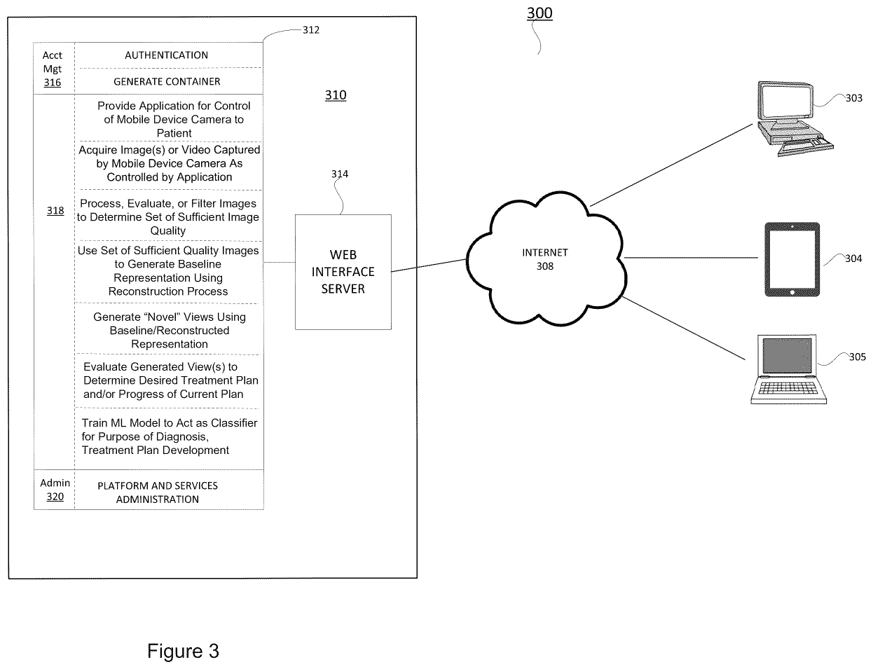

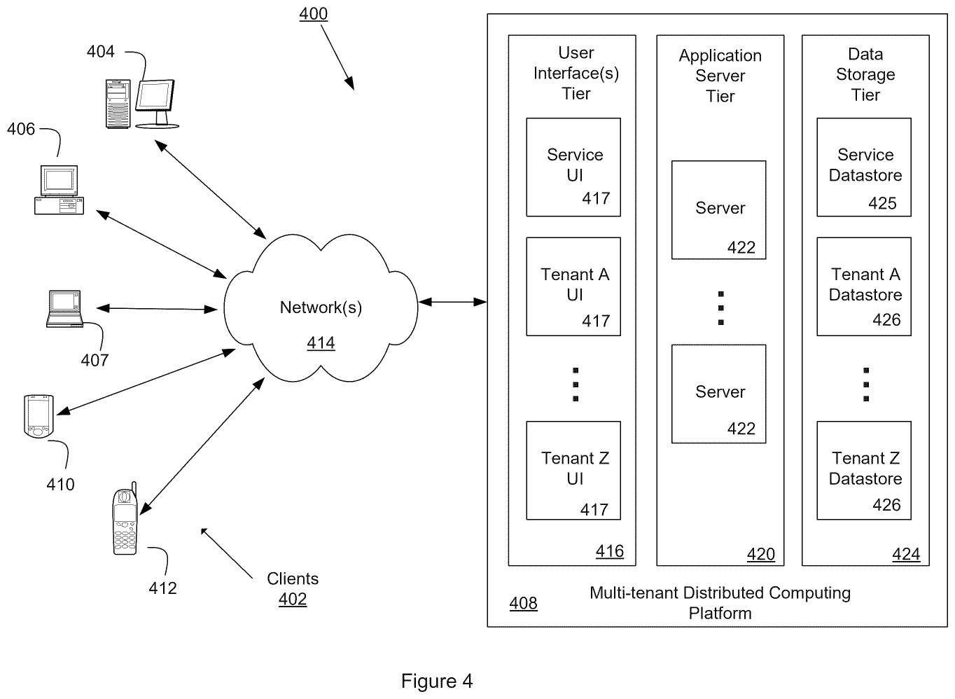

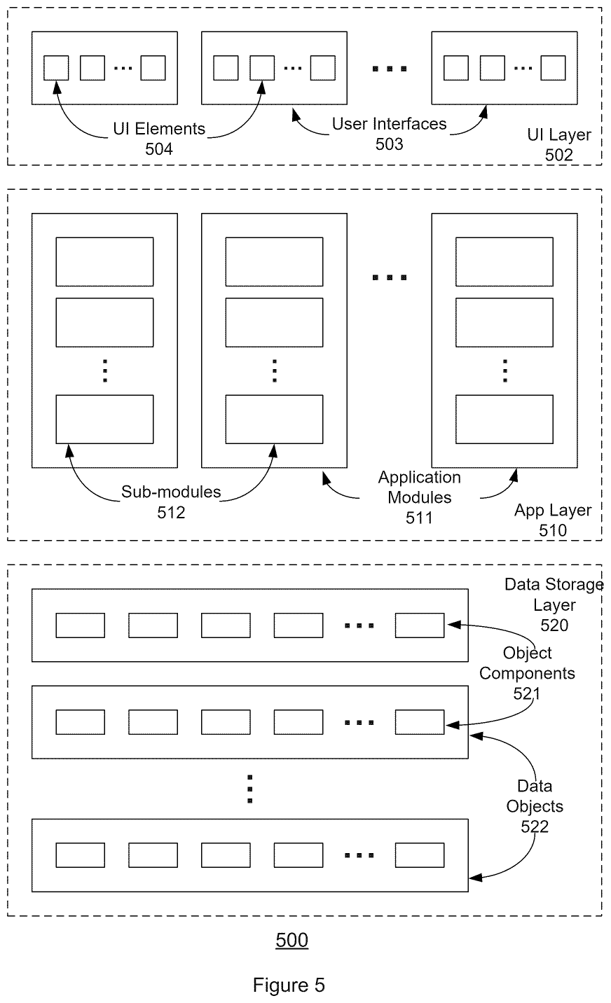

FIG. 3 is a diagram illustrating a SaaS system in which an embodiment of the disclosure may be implemented. FIG. 4 is a diagram illustrating elements or components of an example operating environment in which an embodiment of the disclosure may be implemented. FIG. 5 is a diagram illustrating additional details of the elements or components of the multi-tenant distributed computing service platform of FIG. 4, in which an embodiment of the disclosure may be implemented.

In some embodiments, the system or service(s) disclosed and/or described herein may be implemented as micro-services, processes, workflows, or functions performed in response to requests. The micro-services, processes, workflows, or functions may be performed by a server, data processing element, platform, or system. In some embodiments, the services may be provided by a service platform located “in the cloud”. In such embodiments, the platform is accessible through APIs and SDKs.

The described document processing and evaluation services may be provided as micro-services within the platform for each of multiple users or companies. The interfaces to the micro-services may be defined by REST and GraphQL endpoints. An administrative console may allow users or an administrator to securely access the underlying request and response data, manage accounts and access, and in some cases, modify the processing workflow or configuration.

Note that although FIGS. 3-5 illustrate a multi-tenant or SaaS architecture that may be used for the delivery of business-related or other applications and services to multiple accounts/users, such an architecture may also be used to deliver other types of data processing services and provide access to other applications. For example, such an architecture may be used to provide the document processing and evaluation processes disclosed and/or described herein.

Although in some embodiments, a platform or system of the type illustrated in FIGS. 3-5 may be operated by a 3^{rd} party provider, in other embodiments, the platform may be operated by a provider and a different source may provide the applications or services for users through the platform.

FIG. 3 is a diagram illustrating a system 300 in which an embodiment of the disclosure may be implemented or through which an embodiment of the services disclosed and/or described herein may be accessed. In accordance with the advantages of an application service provider (ASP) hosted business service system (such as a multi-tenant data processing platform), users of the services may comprise individuals, businesses, stores, or organizations, as non-limiting examples. A user may access the services using a suitable client, including but not limited to desktop computers, laptop computers, tablet computers, scanners, or smartphones. In general, a client device having access to the Internet may be used to provide a request or text message requesting a service (such as the processing of a document). Users interface with the service platform across the Internet 308 or another suitable communications network or combination of networks. Non-limiting examples of suitable client devices include desktop computers 303, smartphones 304, tablet computers 305, or laptop computers 306.

System 310, which may be hosted by a third party, may include a set of services 312 and a web interface server 314, coupled as shown in FIG. 3. It is to be appreciated that either or both of services 312 and the web interface server 314 may be implemented on one or more different hardware systems and components, even though represented as singular units in FIG. 3.

Services 312 may include one or more processes, functions, or operations for providing an application to a patient to assist in controlling a mobile device camera to capture a set of images or video, processing a set of images or video captured by the patient's mobile device camera, selecting a set of images for use in a reconstruction process, generating one or more novel views from the reconstruction process, and using the generated novel views to develop and/or assess the progress of a treatment plan for the patient, as non-limiting examples.

In some embodiments, the set of applications or services available to a user may include one or more that perform the functions and methods disclosed and/or described herein. As examples, in some embodiments, the set of applications, functions, operations, or services made available through the platform or system 310 may include:
account management services 316, such as
   a process or service to authenticate a person or entity requesting data processing services (such as credentials, proof of purchase, or verification that the customer has been authorized by a company to use the services provided by the platform);
   a process or service to receive a request for processing of a set of images or video;
   an optional process or service to generate a price for the requested service or a charge against a service contract;
   a process or service to generate a container or instantiation of the requested processes for a user/customer, where the instantiation may be customized for a particular company; and
   other forms of account management services;
a set of processes or services 318, such as
   Provide/Download Application for Control of Mobile Device Camera to Patient;
   Acquire Image(s) or Video Captured by Mobile Device Camera As Controlled by Application;
   Process, Evaluate, or Filter Images to Determine Set of Sufficient Image Quality;
   Use Set of Sufficient Quality Images to Generate Baseline Representation Using Reconstruction Process;
   Generate One or More “Novel” Views Using Baseline/Reconstructed Representation;
   Evaluate Generated View(s) to Determine Desired Treatment Plan and/or Progress of Current Plan;
   Train ML Model to Act as Classifier for Purpose of Diagnosis, Treatment Plan Development;
administrative services 320, such as
   a process or services to enable the provider of the data processing and services and/or the platform to administer and configure the processes and services provided to users.

The platform or system shown in FIG. 3 may be hosted on a distributed computing system made up of at least one, but typically multiple, “servers.” A server is a physical computer dedicated to providing data storage and an execution environment for one or more software applications or services intended to serve the needs of the users of other computers that are in data communication with the server, for instance via a public network such as the Internet. The server, and the services it provides, may be referred to as the “host” and the remote computers, and the software applications running on the remote computers being served may be referred to as “clients.” Depending on the computing service(s) that a server offers it could be referred to as a database server, data storage server, file server, mail server, print server, or web server (as examples).

FIG. 4 is a diagram illustrating elements or components of an example operating environment 400 in which an embodiment of the disclosure may be implemented. As shown, a variety of clients 402 incorporating and/or incorporated into a variety of computing devices may communicate with a multi-tenant service platform 408 through one or more networks 414. For example, a client may incorporate and/or be incorporated into a client application (e.g., software) implemented or executed at least in part by one or more of the computing devices. Examples of suitable computing devices include personal computers, server computers 404, desktop computers 406, laptop computers 407, notebook computers, tablet computers or personal digital assistants (PDAs) 410, smart phones 412, cell phones, and consumer electronic devices incorporating one or more computing device components (such as one or more electronic processors, microprocessors, central processing units (CPU), or controllers). Examples of suitable networks 414 include networks utilizing wired and/or wireless communication technologies and networks operating in accordance with any suitable networking and/or communication protocol (e.g., the Internet).

The distributed computing service/platform (which may also be referred to as a multi-tenant data processing platform) 408 may include multiple processing tiers, including a user interface tier 416, an application server tier 420, and a data storage tier 424. The user interface tier 416 may maintain multiple user interfaces 417, including graphical user interfaces and/or web-based interfaces. The user interfaces may include a default user interface for the service to provide access to applications and data for a user or “tenant” of the service (depicted as “Service UI” in the figure), as well as one or more user interfaces that have been specialized/customized in accordance with user specific requirements (e.g., represented by “Tenant A UI”, . . . , “Tenant Z UI” in the figure, and which may be accessed via one or more APIs).

The default user interface may include user interface components enabling a tenant to administer the tenant's access to and use of the functions and capabilities provided by the service platform. This may include accessing tenant data, launching an instantiation of a specific application, or causing the execution of specific data processing operations, as non-limiting examples. Each application server or processing tier 422 shown in the figure may be implemented with a set of computers and/or components including computer servers and processors, and may perform various functions, methods, processes, or operations as determined by the execution of a software application or set of instructions. The data storage tier 424 may include one or more data stores, which may include a Service Data store 425 and one or more Tenant Data stores 426. Data stores may be implemented with a suitable data storage technology, including structured query language (SQL) based relational database management systems (RDBMS).

Service Platform 408 may be multi-tenant and may be operated by an entity to provide multiple tenants with a set of business-related or other data processing applications, data storage, and functionality. For example, the applications and functionality may include providing web-based access to the functionality used by a business to provide services to end-users, thereby allowing a user with a browser and an Internet or intranet connection to view, enter, process, or modify certain types of information. Such functions or applications are typically implemented by one or more modules of software code/instructions that are maintained on and executed by one or more servers 422 that are part of the platform's Application Server Tier 420. As noted with regards to FIG. 3, the platform system shown in FIG. 4 may be hosted on a distributed computing system made up of at least one, but typically multiple, “servers.”

As mentioned, rather than build and maintain such a platform or system themselves, a business may utilize a platform or system provided by a third party. A third party may implement a business system/platform as described in the context of a multi-tenant platform, where individual instantiations of a business' data processing workflow (such as the image processing and uses disclosed and/or described herein) are provided to users, with each company/business representing a tenant of the platform. One advantage to such multi-tenant platforms is the ability for each tenant to customize their instantiation of the data processing workflow to that tenant's specific business needs or operational methods. Further, each tenant may be a business or entity that uses the multi-tenant platform to provide business services and functionality to multiple users.

FIG. 5 is a diagram illustrating additional details of the elements or components of the multi-tenant distributed computing service platform of FIG. 4, in which an embodiment of the disclosure may be implemented. In general, an embodiment may be implemented using a set of software instructions that are designed to be executed by a suitably programmed processing element (such as a CPU, microprocessor, processor, controller, or computing device). In a complex system such instructions are typically arranged into “modules” with each such module performing a specific task, process, function, or operation. The entire set of modules may be controlled or coordinated in their operation by an operating system (OS) or other form of organizational platform.

The example architecture 500 of a multi-tenant distributed computing service platform illustrated in FIG. 5 includes a user interface layer or tier 502 having one or more user interfaces 503. Examples of such user interfaces include graphical user interfaces and application programming interfaces (APIs). Each user interface may include one or more interface elements 504. For example, users may interact with interface elements to access functionality and/or data provided by application and/or data storage layers of the example architecture. Examples of graphical user interface elements include buttons, menus, checkboxes, drop-down lists, scrollbars, sliders, spinners, text boxes, icons, labels, progress bars, status bars, toolbars, windows, hyperlinks, and dialog boxes. Application programming interfaces may be local or remote and may include interface elements such as parameterized procedure calls, programmatic objects, and messaging protocols.

The application layer 510 may include one or more application modules 511, each having one or more associated sub-modules 512. Each application module 511 or sub-module 512 may correspond to a function, method, process, or operation that is implemented by the module or sub-module (e.g., a function or process related to providing data processing and other services to a user of the platform). Such function, method, process, or operation may include those used to implement one or more aspects of the disclosed system and methods, such as for one or more of the processes, operations, or functions disclosed and/or described with reference to the specification and Figures:
Provide/Download Application for Control of Mobile Device Camera to Patient;
Acquire Image(s) or Video Captured by Mobile Device Camera As Controlled by Application;
Process, Evaluate, or Filter Images to Determine Set of Sufficient Image Quality;
Use Set of Sufficient Quality Images to Generate Baseline Representation Using Reconstruction Process;
Generate One or More “Novel” Views Using Baseline/Reconstructed Representation;
Evaluate Generated View(s) to Determine Desired Treatment Plan and/or Progress of Current Plan;
Train ML Model to Act as Classifier for Purpose of Diagnosis, Treatment Plan Development.

The application modules and/or sub-modules may include any suitable computer-executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language. Each application server (e.g., as represented by element 422 of FIG. 4) may include each application module. Alternatively, different application servers may include different sets of application modules. Such sets may be disjoint or overlapping.

The data storage layer 520 may include one or more data objects 522 each having one or more data object components 521, such as attributes and/or behaviors. For example, the data objects may correspond to tables of a relational database, and the data object components may correspond to columns or fields of such tables. Alternatively, or in addition, the data objects may correspond to data records having fields and associated services. Alternatively, or in addition, the data objects may correspond to persistent instances of programmatic data objects, such as structures and classes. Each data store in the data storage layer may include each data object. Alternatively, different data stores may include different sets of data objects. Such sets may be disjoint or overlapping.

Note that the example computing environments depicted in FIGS. 3-5 are not intended to be limiting examples. Further environments in which an embodiment may be implemented in whole or in part include devices (including mobile devices), software applications, systems, apparatuses, networks, SaaS platforms, IaaS (infrastructure-as-a-service) platforms, or other configurable components that may be used by multiple users for data entry, data processing, application execution, or data review (as non-limiting examples).

Embodiments as disclosed and/or described herein can be implemented in the form of control logic using computer software in a modular or integrated manner. Based on the disclosure and teachings provided herein, a person of ordinary skill in the art will know and appreciate other ways and/or methods to implement the present invention using hardware and a combination of hardware and software.

The disclosure includes the following clauses and embodiments:

Clause 1. A method for performing an orthodontic treatment, comprising: receiving an orthodontic treatment plan comprising a plurality of treatment stages to move teeth of a patient from a first teeth arrangement towards a second teeth arrangement, each of the plurality of treatment stages including associated with a respective teeth arrangement of the teeth of the patient; receiving one or more first images of the teeth of a patient captured at a first time when the teeth of the patient are in a first teeth arrangement, the first images having been captured from one or more first camera positions, orientations, or settings; receiving one or more second images of the teeth of the patient captured at a second time when the teeth of the patient are in a second teeth arrangement, the second time being different from the first time, the second images having been captured from one or more second camera positions, orientations, or settings; and generating, based on one or more of the first or second images, a novel image of the teeth in the first or second teeth arrangement, the novel image representing the teeth in either the first or second teeth arrangement as viewed from a different camera position, orientation, or setting than that of the one or more first or second images.

Clause 2. The method of clause 1, further comprising: displaying the novel image and the one or more of the first or second images used to generate the novel image.

Clause 3. The method of clause 1, wherein the novel image and the and the one or more of the first or second images used to generate the novel image are displayed at the same time.

Clause 4. The method of clause 1, wherein the novel image is generated as viewed from a position and orientation that matches a position and orientation of an image of the one or more first images or the one or more second images.

Clause 5. The method of clause 1, wherein the novel image is generated as viewed from a position and orientation that does not match a position and orientation of the one or more first images or the one or more second images.

Clause 6. The method of clause 1, further comprising: accessing an orthodontic treatment plan comprising a plurality of treatment stages to move teeth of the patient from a first teeth arrangement towards a second teeth arrangement, each of the plurality of treatment stages associated with a respective teeth arrangement of the teeth of the patient; and evaluating the progress of the treatment plan by comparing the novel image and the one or more of the first or second images used to generate the novel image.

Clause 7. The method of clause 1, wherein generating, based on one or more of the first or second images, a novel image of the teeth in the first or second teeth arrangement, the novel image representing the teeth in either the first or second teeth arrangement as viewed from a different camera position and orientation than that of the one or more first or second images further comprises using one or more of Neural Radiance Fields, Gaussian Splatting, Smart Image stitching, Gaussian Splatting or a Fine-tuned diffusion model.

Clause 8. The method of clause 1, wherein the first time is prior to implementation of the treatment plan, and the second time is after implementation of at least a portion of the treatment plan.

Clause 9. The method of clause 1, wherein the different camera position and orientation correspond to that of one of the first or second images.

Clause 10. The method of clause 1, wherein the one or more first images or the one or more second images are obtained from a video provided by the patient.

Clause 11. The method of clause 1, wherein prior to generating, based on one or more of the first or second images, a novel image of the teeth in the first or second teeth arrangement, the method comprises evaluating the quality of one or more of the first or second images.

Clause 12. The method of clause 7, wherein evaluating the quality of one or more of the first or second images further comprises using a trained model to evaluate the quality.

Clause 13. The method of clause 1, wherein the first images of the teeth are captured at a plurality of exposure settings.

Clause 14. The method of clause 1, wherein the first images of the teeth are captured at a plurality of focus settings.

Clause 15. The method of clause 7, wherein evaluating the quality of one or more of the first or second images further comprises determining a blur metric.

Clause 16. The method of clause 8, further comprising, displaying the uncaptured 2D image of the teeth in the second teeth arrangement.

Clause 17. The method of clause 1, wherein the first time is during a first treatment stage and this second time is during a second treatment stage.

Clause 18. The method of clause 1, wherein the treatment stage comprises a time prior to beginning the orthodontic treatment.

Clause 19. The method of clause 1, wherein the second 2D image includes a plurality of still images.

Clause 20. The method of clause 1, wherein the second images includes a video.

Clause 21. The method of clause 1, further comprising: selecting an image from the second images, wherein a first of the plurality of camera positions and orientations associated with the selected image from the first images more closely corresponds to the first of the positions and orientations associated with the first images than other of the plurality of camera positions and orientations.

Clause 22. The method of clause 21, further comprising: displaying the selected image form the first images while displaying the second image.

Clause 23. The method of clause 1, wherein generating the second image includes generating a Gaussian Splat image based on the second image.

Clause 24. The method of clause 1, wherein generating the second image includes: generating Gaussian Splatting 3D representation of the teeth of the patient based on the second image; and rendering a 2D image of the 3D representation of the teeth from the first of the camera positions and orientations associated with the first image to generate the second 2D image of the teeth.

Clause 25. The method of clause 1, wherein generating the second 2D image includes: generating a neural radiance field of the teeth of the patient based on the second 2D image; and rendering, using the neural radiance field, a 2D image of a 3D representation of the teeth from the first of the camera positions and orientations associated with the first 2D image to generate the second 2D image of the teeth.

Clause 26. The method of clause 1, further comprising determining that the second camera positions and orientations are different from the first camera positions and orientations.

Clause 27. A method for orthodontic treatment, the method comprising: receiving an orthodontic treatment plan comprising a plurality of treatment stages to move teeth of a patient from a first teeth arrangement towards a second teeth arrangement, each of the plurality of treatment stages including a respective arrangement of the teeth of the patient; receiving first 2D images of the teeth of the patient captured at a first time; receiving second 2D images of the teeth of the patient captured at a second time corresponding to a stage of the orthodontic treatment plan, the second 2D images being captured from a plurality of camera positions and orientations and with a plurality of image properties; selecting a subset of the second 2D images based on the image properties; and combining the subset of second 2D images to generate third 2D images comprising a plurality of 2D images at the plurality of camera positions and orientations; and comparing a position of the teeth of the patient in the first 2D images with the position of the teeth in the third 2D images.

Clause 28. The method of clause 27, wherein the plurality of image properties includes image exposure.

Clause 29. The method of clause 28, wherein combining the subset of second 2D images includes aligning the subset of 2D images and tone mapping the 2D images, wherein the plurality of 2D images at the plurality of camera positions and orientations include a plurality of tone mapped 2D images at the plurality of camera positions and orientations.

Clause 30. The method of clause 27, wherein the plurality of image properties includes image focal planes.

Clause 31. The method of clause 30, wherein combining the subset of second 2D images includes aligning the subset of 2D images and combining in focus portions of the subset of second 2D images to generate the plurality of 2D images at the plurality of camera positions and orientations.

Clause 32. The method of clause 27, wherein the plurality of image properties includes depth of field.

Clause 33. The method of clause 32, wherein combining the subset of second 2D images includes aligning the subset of 2D images and combining in focus portions of the subset of second 2D images to generate the plurality of 2D images at the plurality of camera positions and orientations.

Clause 34. The method of clause 27, wherein the first time is before dental treatment has started and the second time is after treatment has started.

Clause 35. A method for orthodontic treatment, the method comprising: receiving first 2D images of the teeth of the patient, the first 2D images being captured from a plurality of camera positions and orientations and corresponding to a stage of the orthodontic treatment plan; assessing a quality of the first 2D images by iteratively: altering an aspect of each of the first 2D images; determining a metric for the altered aspect of a plurality of portions of each of the first 2D images; aggregating the determined metric for each of the plurality of portions of each of the first 2D images to generate an aggregated metric; and determining a quality metric for the image based on the aggregated metric over the iterations.

Clause 36. The method of clause 35, wherein: the aspect is the blurriness of the image.

Clause 37. The method of clause 36, wherein: wherein altering includes applying a blur filter to the image.

Clause 38. The method of clause 37, wherein: the blur filter is a Gaussian blur filter. Clause 39. The method of clause 37, wherein: wherein the metric is a blur metric.

Clause 40. The method of clause 39, wherein determining a metric for the altered aspect of a plurality of portions of each of the first 2D images includes applying a Laplacian filter to the blurred image.

Clause 41. The method of clause 40, wherein determining a quality metric for the image based on the aggregated metric over the iterations includes determining a slope of the aggregated blur metric over the first two iterations.

Clause 42. The method of clause 40, wherein determining a quality metric for the image based on the aggregated metric over the iterations includes determining a value of the aggregated blur metric after at least 10 iterations.

Clause 43. The method of clause 40, wherein determining a quality metric for the image based on the aggregated metric over the iterations includes determining a value of an area of a curve of the aggregated blur metric verses iterations after at least 10 iterations.

Clause 44. A method for orthodontic treatment, the method comprising: receiving first 2D images of the teeth of the patient; receiving labels from a plurality of labelers indicating a location of dental appliance structures in first 2D images; comparing the location for each of the plurality of dental appliance structures from each of the plurality of labelers; and determining an image quality metric for each of the first 2D images based on the comparison.

Clause 45. The method of clause 44, wherein the comparison includes comparing the presence or absence of a dental appliance structure on a particular tooth for each of the labelers.

Clause 46. The method of clause 45, wherein determining the image quality metric is based on a degree of agreement of the presence or absence of a dental appliance structure on a particular tooth for each of the labelers.

Clause 47. The method of clause 46, wherein greater agreement indicates a higher quality image.

Clause 48. The method of clause 47, wherein determining an image quality metric for each of the first 2D images based on the comparison includes determining an image quality metric of a plurality of portions of each of the first 2D images based on the comparison.

Clause 49. The method of clause 45, wherein the labels from a plurality of labelers include labels for dental appliance structures on anterior teeth and posterior teeth.

Clause 50. The method of clause 49, wherein determining the image quality metric of the plurality of portions of each of the first 2D images based on the comparison determining a degree of agreement of the presence or absence of dental appliance structures on anterior teeth and posterior teeth.

Clause 51. The method of clause 50, wherein the comparing the location for each of the plurality of dental appliance structures from each of the plurality of labelers includes comparing the location for each of the plurality of dental appliance structures from each of the plurality of labelers with locations of appliance structures in a first stage of the orthodontic treatment plan.

Clause 52. The method of clause 45, wherein the quality metric for portion of the image including anterior teeth is based on the degree of agreement of the presence or absence of dental appliance structures on anterior teeth and the quality metric for portion of the image including posterior teeth is based on the degree of agreement of the presence or absence of dental appliance structures on posterior teeth.

Clause 53. The method of clause 44, wherein the appliance structures are attachments.

Clause 54. The method of clause 44, wherein the appliance structures are structures attached to the patient's tissue.

Clause 55. The method of clause 44, wherein the appliance structures are structures on an orthodontic appliance worn by the patient when the first 2D images were captured.

Clause 56. The method of clause 44, wherein the labels include a confidence value that indicates the confidence the labeler had with their determining that an appliance structure is at the labeled location.

Clause 57. The method of clause 44, wherein the labelers are human or algorithmic.

Clause 58. A method for performing an orthodontic treatment, comprising: receiving a first set of images of a patient's mouth captured using a mobile device; evaluating the first set of images to produce a second set of images for use in a reconstruction process; using the second set of images as inputs to a reconstruction process to generate a baseline representation for generating a novel view or views of the patient's mouth; generating one or more novel views from the generated baseline representation; and evaluating the generated novel view or views to determine a desired treatment plan for the patient or to determine a current state of a patient's teeth and monitor progress of a treatment plan.

Clause 59. The method of clause 58, wherein instead of the set of images, a video is captured using the mobile device, and the method further comprises processing the video to generate the first set of images.

Clause 60. The method of clause 58, further comprising: training a machine learning model to operate as a classifier using multiple generated views; and operating the trained model to perform a diagnostic or evaluation function.

Clause 61. The method of clause 58, wherein the reconstruction process comprises one or more of: Neural Radiance Fields (NeRFs); Gaussian Splatting; Image stitching; or a diffusion model.

Clause 62. The method of clause 58, further comprising using an application installed in the mobile device to control an operation of the camera or mobile device during capture of the set of images.

Clause 63. The method of clause 62, wherein the application controls one or more of the camera depth of field, camera exposure setting, or illumination.

Clause 64. The method of clause 62, wherein the application provides a user interface display or audio segment to assist the patient in capturing the images.

Clause 65. The method of clause 64, wherein sensor data from the mobile device is used to provide guidance to the patient in positioning the mobile device or altering the environment in which the images are collected.

Clause 66. The method of clause 65, wherein the sensor data is one or more of a light sensor or gyroscope in the mobile device.

Clause 67. The method of clause 65, wherein the sensor data applicable to each image is associated with the image.

Clause 68. The method of clause 67, wherein the sensor data is used to adjust the appearance of one or more of the images.

Clause 69. The method of clause 58, wherein evaluating the provided images to produce a second set of images for use in a reconstruction process further comprises determining a quality of one or more of the provided images and removing images of insufficient quality before producing the second set of images.

Clause 70. The method of clause 69, wherein determining the quality of one or more of the provided images further comprises one or more of: determining a degree of blur in an image; performing a comparison to previously obtained images; or operating a trained model to generate an indication of the relative quality or utility of an image.

Clause 71. The method of clause 58, further comprising: receiving an image of the patient's mouth taken at a later time than the first set of images; accessing a stored version of the baseline representation; using the baseline representation to generate a novel view of the patient's mouth, the generated novel view having one or more of camera position, camera orientation, camera setting, or illumination characteristics of the received image; comparing the generated novel view to the received image; and based on the comparison, determining whether to maintain or alter a treatment plan for the patient.

Clause 72. The method of clause 71, wherein the determination of whether to maintain or alter a treatment plan for the patient is performed by one or more of a dentist, orthodontist, or trained model.

Clause 73. The method of clause 70, wherein the trained model is trained using a set of images and associated labels, and further, wherein the accuracy of the labels is determined using a model trained to identify errors in labeling by comparing more than a single source of the set of images and associated labels.

Clause 74. A system for performing an orthodontic treatment, comprising: an application to control one or more aspects of a camera of a mobile device; a remote platform or server for receiving a first set of images of a patient's mouth captured by the camera, and in response evaluating the first set of images to produce a second set of images for use in a reconstruction process; using the second set of images as inputs to a reconstruction process to generate a baseline representation for generating a novel view or views of the patient's mouth; generating one or more novel views from the generated baseline representation; and evaluating the generated novel view or views to determine a desired treatment plan for the patient or to determine a current state of a patient's teeth and monitor progress of a treatment plan.

Clause 75. The system of clause 74, wherein the application controls one or more of the camera depth of field, camera exposure setting, use of a panoramic setting, or illumination.

Clause 76. The system of clause 75, wherein the application provides a user interface display or audio segment to assist the patient in capturing the images.

Clause 77. The system of clause 74, wherein the remote platform or server further operates to: receive an image of the patient's mouth taken at a later time than the first set of images; access a stored version of the baseline representation; use the baseline representation to generate a novel view of the patient's mouth, the generated novel view having one or more of camera position, camera orientation, camera setting, or illumination characteristics of the received image; compare the generated novel view to the received image; and based on the comparison, determine whether to maintain or alter a treatment plan for the patient.

Clause 78. A method for performing an orthodontic treatment, comprising: receiving a first set of images of a patient's mouth captured using a mobile device; evaluating the first set of images to produce a second set of images for use in a reconstruction process; using the second set of images as inputs to a reconstruction process to generate a baseline representation for generating a novel view or views of the patient's mouth; generating one or more novel views from the generated baseline representation; and evaluating the generated novel view or views to determine a desired treatment plan for the patient or to determine a current state of a patient's teeth and monitor progress of a treatment plan.

Clause 79. A method for use with orthodontic treatment, comprising: receiving a first plurality of images of a patient's dentition, the first plurality of images captured at a plurality of different positions, orientations, and focus distances; determining a quality of each of the images of the first plurality of images; determining, based on quality of each of the images, one or more images captured at different focus distances to combine; combining the one or more images captured at different focus distances to generate a focus stacked image.

Clause 80. The method of clause 79, wherein the focus stacked image is an image in the first set of images, one or more second images of the teeth of a patient, one or more first images of the teeth of a patient, first 2D images, or second 2D images of any one of clauses 1-78.

Clause 81. The method of clause 79, further comprising: comparing the quality of the focus stacked image to the one or more images captured at different focus distances; and selecting the focus stacked image or the one or more images captured at different focus distances based on the comparison of the quality.

Clause 82. A system for use in performing an orthodontic treatment, comprising: a processor; and memory comprising instructions, that when executed by the processor, cause the system to: access one or more first image data of teeth of a patient captured at a first time when the teeth of the patient are in a first teeth arrangement, the first image data having been captured from one or more first camera positions, orientations, or settings; access one or more second image data of the teeth of the patient captured at a second time when the teeth of the patient are in a second teeth arrangement, the second time being different from the first time, the second image data having been captured from one or more second camera positions, orientations, or settings; and generate, based on one or more of the first or second image data, a novel image data of the teeth in the first or second teeth arrangement, the novel image data representing the teeth in either the first or second teeth arrangement as viewed from a different camera position, orientation, or setting than that of the one or more first or second image data.

Clause 83. The system for use in performing an orthodontic treatment of clause 82, wherein the instructions, when executed by the processor, further cause the system to: receive an orthodontic treatment plan comprising a plurality of treatment stages to move teeth of a patient from a first teeth arrangement towards a second teeth arrangement, each of the plurality of treatment stages including associated with a respective teeth arrangement of the teeth of the patient.

Clause 84. The system for use in performing an orthodontic treatment of clause 82, wherein the first and second image data are first still images and second still images.

Clause 85. The system for use in performing an orthodontic treatment of clause 84, wherein the novel image data is a new still image.

Clause 86. The system for use in performing an orthodontic treatment of clause 85, wherein the instructions, that when executed by the processor, further cause the system to: display, at the same time, the new still image and the one or more of the first or second still images used to generate the new still image.

Clause 87. The system for use in performing an orthodontic treatment of clause 86, wherein the new still image is generated as viewed from a position and orientation that matches a position and orientation of an image of the first still images or the second still images.

Clause 88. The system for use in performing an orthodontic treatment of clause 86, wherein the new still image is generated as viewed from a position and orientation that does not match a position and orientation of the first still images or the second still images.

Clause 89. The system for use in performing an orthodontic treatment of clause 82, wherein the first and second image data are first videos and second videos.

Clause 90. The system for use in performing an orthodontic treatment of clause 89, wherein the novel image data is a new video.

Clause 91. The system for use in performing an orthodontic treatment of clause 90, wherein the instructions, that when executed by the processor, further cause the system to: display, at the same time, the new video and the one or more of the first or second videos used to generate the new video.

Clause 92. The system for use in performing an orthodontic treatment of clause 91, wherein the new video is generated as viewed from a position and orientation that matches a position and orientation of the first videos or the second videos.

Clause 93. The system for use in performing an orthodontic treatment of clause 91, wherein the new video is generated as viewed from a position and orientation that does not match a position and orientation of the first videos or the second videos.

Clause 94. The system for use in performing an orthodontic treatment of clause 82, wherein the instructions, that when executed by the processor, further cause the system to: access an orthodontic treatment plan comprising a plurality of treatment stages to move teeth of the patient from a first teeth arrangement towards a second teeth arrangement, each of the plurality of treatment stages associated with a respective teeth arrangement of the teeth of the patient; and evaluate progress of the orthodontic treatment plan by comparing the novel image data and the one or more of the first or second image data used to generate the novel image data.

Clause 95. The system for use in performing an orthodontic treatment of clause 82, wherein the generation, based on one or more of the first or second image data, the novel image data of the teeth in the first or second teeth arrangement, the novel image data representing the teeth in either the first or second teeth arrangement as viewed from a different camera position and orientation than that of the one or more first or second image data further uses one or more of Neural Radiance Fields, Gaussian Splatting, smart image stitching, gaussian splatting or a fine-tuned diffusion model.

Clause 96. The system for use in performing an orthodontic treatment of clause 83, wherein the first time is prior to implementation of the orthodontic treatment plan, and the second time is after implementation of at least a portion of the orthodontic treatment plan.

Clause 97. A method for use in performing an orthodontic treatment, comprising: capturing one or more first image data of teeth of a patient captured at a first time when the teeth of the patient are in a first teeth arrangement, the first image data having been captured from one or more first camera positions, orientations, or settings; capturing one or more second image data of the teeth of the patient captured at a second time when the teeth of the patient are in a second teeth arrangement, the second time being different from the first time, the second image data having been captured from one or more second camera positions, orientations, or settings; sending the first and second image data to a remote server device for processing; and receiving, from the remote server device, a novel image data of the teeth in the first or second teeth arrangement, wherein the novel image data is generated based on the first and second image data, the novel image data representing the teeth in either the first or second teeth arrangement as viewed from a different camera position, orientation, or setting than that of the one or more first or second image data.

Clause 98. The method of clause 97, displaying, at the same time, the novel image data and the and the one or more of the first or second image data used to generate the novel image data.

Clause 99. The method of clause 98, wherein the novel image data is generated as viewed from a position and orientation that matches a position and orientation of an image of the one or more first image data or the one or more second image data.

Clause 100. The method of clause 97, wherein the first time is prior to implementation of an orthodontic treatment plan, and the second time is after implementation of at least a portion of the orthodontic treatment plan.

Clause 101. The method of clause 97, wherein the different camera position and orientation correspond to the position and orientation of one of the first or second image data.

The software components, processes, or functions disclosed and/or described in this application may be implemented as software code to be executed by a processor using a suitable computer language such as Python, Java, JavaScript, C, C++, or Perl using conventional or object-oriented techniques. The software code may be stored as a series of instructions or commands in (or on) a non-transitory computer-readable medium, such as a random-access memory (RAM), a read only memory (ROM), a magnetic medium such as a hard-drive, or an optical medium such as a CD-ROM. In this context, a non-transitory computer-readable medium is a medium suitable for the storage of data or an instruction set aside from a transitory waveform. Such computer readable medium may reside on or within a single computational apparatus and may be present on or within different computational apparatuses within a system or network.

According to one example implementation, the term processing element or processor, as used herein, may be a central processing unit (CPU), or conceptualized as a CPU (such as a virtual machine). In this example implementation, the CPU or a device in which the CPU is incorporated may be coupled, connected, and/or in communication with one or more peripheral devices, such as a display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, synchronous dynamic random access memory (SDRAM), or similar devices or forms of memories based on similar technologies. Such computer-readable storage media allow the processing element or processor to access computer-executable process steps and application programs, stored on removable and non-removable memory media, to off-load data from a device or to upload data to a device. As mentioned, with regards to the embodiments disclosed and/or described herein, a non-transitory computer-readable medium may include a structure, technology, or method apart from a transitory waveform or similar medium.

Example embodiments of the disclosure are described herein with reference to block diagrams of systems, and/or flowcharts or flow diagrams of functions, operations, processes, or methods. One or more blocks of the block diagrams, or one or more stages or steps of the flowcharts or flow diagrams, and combinations of blocks in the block diagrams and combinations of stages or steps of the flowcharts or flow diagrams may be implemented by computer-executable program instructions. In some embodiments, one or more of the blocks, or stages or steps may not necessarily need to be performed in the order presented or may not necessarily need to be performed at all.

The computer-executable program instructions may be loaded onto a general-purpose computer, a special purpose computer, a processor, or other programmable data processing apparatus to produce a specific example of a machine. The instructions that are executed by the computer, processor, or other programmable data processing apparatus create means for implementing one or more of the functions, operations, processes, or methods disclosed and/or described herein. The computer program instructions may be stored in (or on) a computer-readable memory that may direct a computer or other programmable data processing apparatus to function in a specific manner, such that the instructions stored in (or on) the computer-readable memory produce an article of manufacture including instruction means that when executed implement one or more of the functions, operations, processes, or methods disclosed and/or described herein.

While embodiments of the disclosure have been described in connection with what is presently considered to be the most practical approach and technology, the embodiments are not limited to the disclosed implementations. Instead, the disclosed implementations are intended to include and cover modifications and equivalent arrangements included within the scope of the appended claims. Although specific terms are employed herein, they are used in a generic and descriptive sense only and not for purposes of limitation.

This written description uses examples to describe one or more embodiments of the disclosure, and to enable a person skilled in the art to practice the disclosed approach and technology, including making and using devices or systems and performing the associated methods. The patentable scope of the disclosure is defined by the claims, and may include other examples that occur to those skilled in the art. Such other examples are intended to be within the scope of the claims if they have structural and/or functional elements that do not differ from the literal language of the claims, or if they include structural and/or functional elements with insubstantial differences from the literal language of the claims.

All references, including publications, patent applications, and patents, cited herein are hereby incorporated by reference to the same extent as if each reference was individually and specifically indicated to be incorporated by reference and/or was set forth in its entirety herein.

The use of the terms “a” and “an” and “the” and similar references in the specification and in the claims are to be construed to cover both the singular and the plural, unless otherwise indicated herein or clearly contradicted by context. The terms “having,” “including,” “containing” and similar references in the specification and in the claims are to be construed as open-ended terms (e.g., meaning “including, but not limited to,”) unless otherwise noted.

Recitation of ranges of values herein are intended to serve as a shorthand method of referring individually to each separate value inclusively falling within the range, unless otherwise indicated herein, and each separate value is incorporated into the specification as if it were individually recited herein. Method steps or stages disclosed and/or described herein may be performed in any suitable order unless otherwise indicated herein or clearly contradicted by context.

The use of examples or exemplary language (e.g., “such as”) herein, is intended to illustrate embodiments of the disclosure and does not pose a limitation to the scope of the claims unless otherwise indicated. No language in the specification should be construed as indicating any non-claimed element as essential to each embodiment of the disclosure.

As used herein (i.e., the claims, figures, and specification), the term “or” is used inclusively to refer items in the alternative and in combination.

Different arrangements of the elements, structures, components, or steps illustrated in the figures or described herein, as well as components and steps not shown or described are possible. Similarly, some features and sub-combinations are useful and may be employed without reference to other features and sub-combinations. Embodiments have been described for illustrative and not for restrictive purposes, and alternative embodiments may become apparent to readers of the specification. Accordingly, the disclosure is not limited to the embodiments described in the specification or depicted in the figures, and modifications may be made without departing from the scope of the appended claims.

One or more embodiments of the disclosed subject matter are described herein with specificity to meet statutory requirements, but this description does not limit the scope of the claims. The claimed subject matter may be embodied in other ways, may include different elements or steps, and may be used in conjunction with other existing or later developed technologies. This description should not be interpreted as implying any required order or arrangement among or between various steps or elements except when the order of individual steps or arrangement of elements is explicitly noted as being required.

Embodiments of the disclosure will be described more fully herein with reference to the accompanying drawings, which form a part hereof, and which show, by way of illustration, exemplary embodiments by which the disclosure may be practiced. The disclosure may, however, be embodied in different forms and should not be construed as limited to the embodiments set forth herein; rather, these embodiments are provided so that this disclosure will satisfy the statutory requirements and convey the scope of the disclosure to those skilled in the art.

Among others, the subject matter of the disclosure may be embodied in whole or in part as a system, as one or more methods, or as one or more devices. Embodiments may take the form of a hardware implemented embodiment, a software implemented embodiment, or an embodiment combining software and hardware aspects. For example, in some embodiments, one or more of the operations, functions, processes, or methods described herein may be implemented by one or more suitable processing elements (such as a processor, microprocessor, co-processor, CPU, GPU, TPU, QPU, or controller, as non-limiting examples) that is part of a client device, server, network element, remote platform (such as a SaaS platform), an “in the cloud” service, or other form of computing or data processing system, device, or platform.

The processing element or elements may be programmed with a set of executable instructions (e.g., software instructions), where the instructions may be stored in (or on) one or more suitable non-transitory data storage elements. In some embodiments, the set of instructions may be conveyed to a user through a transfer of instructions or an application that executes a set of instructions (such as over a network, e.g., the Internet). In some embodiments, a set of instructions or an application may be utilized by an end-user through access to a SaaS platform or a service provided through such a platform.

In some embodiments, the systems and methods disclosed herein may provide services through a SaaS or multi-tenant platform. The platform provides access to multiple entities, each with a separate account and associated data storage. Each account may correspond to a dentist or orthodontist, a patient, an entity, a set or category of entities, a set or category of patients, an insurance company, or an organization, for example. Each account may access one or more services, a set of which are instantiated in their account, and which implement one or more of the methods or functions disclosed and/or described herein.

In some embodiments, one or more of the operations, functions, processes, or methods described herein may be implemented by a specialized form of hardware, such as a programmable gate array, application specific integrated circuit (ASIC), or the like. Note that an embodiment of the inventive methods may be implemented in the form of an application, a sub-routine that is part of a larger application, a “plug-in”, an extension to the functionality of a data processing system or platform, or other suitable form. The following detailed description is, therefore, not to be taken in a limiting sense.

What is claimed is:

1. A system for use in performing an orthodontic treatment, comprising:
a processor; and
memory comprising instructions, that when executed by the processor, cause the system to:
access one or more first image data of teeth of a patient captured at a first time when the teeth of the patient are in a first teeth arrangement, the first image data having been captured from one or more first camera positions, orientations, or settings;
access one or more second image data of the teeth of the patient captured at a second time when the teeth of the patient are in a second teeth arrangement, the second time being different from the first time, the second image data having been captured from one or more second camera positions, orientations, or settings; and
generate, based on one or more of the first or second image data, a novel image data of the teeth in the first or second teeth arrangement, the novel image data representing the teeth in either the first or second teeth arrangement as viewed from a different camera position, orientation, or setting than that of the one or more first or second image data.

2. The system for use in performing an orthodontic treatment of claim 1, wherein the instructions, when executed by the processor, further cause the system to:
receive an orthodontic treatment plan comprising a plurality of treatment stages to move teeth of a patient from a first teeth arrangement towards a second teeth arrangement, each of the plurality of treatment stages including associated with a respective teeth arrangement of the teeth of the patient.

3. The system for use in performing an orthodontic treatment of claim 1, wherein the first and second image data are first still images and second still images.

4. The system for use in performing an orthodontic treatment of claim 3, wherein the novel image data is a new still image.

5. The system for use in performing an orthodontic treatment of claim 4, wherein the instructions, that when executed by the processor, further cause the system to:
display, at the same time, the new still image and the one or more of the first or second still images used to generate the new still image.

6. The system for use in performing an orthodontic treatment of claim 5, wherein the new still image is generated as viewed from a position and orientation that matches a position and orientation of an image of the first still images or the second still images.

7. The system for use in performing an orthodontic treatment of claim 5, wherein the new still image is generated as viewed from a position and orientation that does not match a position and orientation of the first still images or the second still images.

8. The system for use in performing an orthodontic treatment of claim 1, wherein the first and second image data are first videos and second videos.

9. The system for use in performing an orthodontic treatment of claim 8, wherein the novel image data is a new video.

10. The system for use in performing an orthodontic treatment of claim 9, wherein the instructions, that when executed by the processor, further cause the system to:
display, at the same time, the new video and the one or more of the first or second videos used to generate the new video.

11. The system for use in performing an orthodontic treatment of claim 10, wherein the new video is generated as viewed from a position and orientation that matches a position and orientation of the first videos or the second videos.

12. The system for use in performing an orthodontic treatment of claim 10, wherein the new video is generated as viewed from a position and orientation that does not match a position and orientation of the first videos or the second videos.

13. The system for use in performing an orthodontic treatment of claim 1, wherein the instructions, that when executed by the processor, further cause the system to:
access an orthodontic treatment plan comprising a plurality of treatment stages to move teeth of the patient from a first teeth arrangement towards a second teeth arrangement, each of the plurality of treatment stages associated with a respective teeth arrangement of the teeth of the patient; and
evaluate progress of the orthodontic treatment plan by comparing the novel image data and the one or more of the first or second image data used to generate the novel image data.

14. The system for use in performing an orthodontic treatment of claim 1, wherein the generation, based on one or more of the first or second image data, the novel image data of the teeth in the first or second teeth arrangement, the novel image data representing the teeth in either the first or second teeth arrangement as viewed from a different camera position and orientation than that of the one or more first or second image data further uses one or more of Neural Radiance Fields, Gaussian Splatting, smart image stitching, gaussian splatting or a fine-tuned diffusion model.

15. The system for use in performing an orthodontic treatment of claim 2, wherein the first time is prior to implementation of the orthodontic treatment plan, and the second time is after implementation of at least a portion of the orthodontic treatment plan.

16. A method for use in performing an orthodontic treatment, comprising:
capturing one or more first image data of teeth of a patient captured at a first time when the teeth of the patient are in a first teeth arrangement, the first image data having been captured from one or more first camera positions, orientations, or settings;
capturing one or more second image data of the teeth of the patient captured at a second time when the teeth of the patient are in a second teeth arrangement, the second time being different from the first time, the second image data having been captured from one or more second camera positions, orientations, or settings;
sending the first and second image data to a remote server device for processing; and
receiving, from the remote server device, a novel image data of the teeth in the first or second teeth arrangement, wherein the novel image data is generated based on the first and second image data, the novel image data representing the teeth in either the first or second teeth arrangement as viewed from a different camera position, orientation, or setting than that of the one or more first or second image data.

17. The method of claim 16, displaying, at the same time, the novel image data and the and the one or more of the first or second image data used to generate the novel image data.

18. The method of claim 17, wherein the novel image data is generated as viewed from a position and orientation that matches a position and orientation of an image of the one or more first image data or the one or more second image data.

19. The method of claim 16, wherein the first time is prior to implementation of an orthodontic treatment plan, and the second time is after implementation of at least a portion of the orthodontic treatment plan.

20. The method of claim 16, wherein the different camera position and orientation correspond to the position and orientation of one of the first or second image data.
